(19) United States
(12) Patent Application Publication
(10) Pub. No.: US 2026/0258073 A1
(43) Pub. Date: Sep. 3, 2026

(54) CRYSTALLINE FORMS OF A NEUROACTIVE STEROID

(71) Applicant: SAGE THERAPEUTICS, INC., Cambridge, MA (US)

(72) Inventors: Paul Watson, Cambridge, MA (US); Reiko Kawahama, South Plainfield, NJ (US); Ji Zhang, South Plainfield, NJ (US); Peter Wade, South Plainfield, NJ (US); Jian Wang, South Plainfield, NJ (US); Jiaqi Yao, Cranbury, NJ (US); Shanming Kuang, Cranbury, NJ (US)

(21) Appl. No.: 18/877,746

(22) PCT Filed: Jun. 23, 2023
(86) PCT No.: PCT/US2023/026143

§ 371 Date: Dec. 20, 2024

Related U.S. Application Data
(60) Provisional application No. 63/355,238, filed on Jun. 24, 2022.

(51) Int. Cl. C07J 9/00

(52) U.S. Cl. CPC C07J 9/00; C07B 2200/13

(57) ABSTRACT

The disclosure relates to Compound 1 and crystalline solid forms and solid dosage forms thereof. The disclosure also provides methods of preparing and methods of using said crystalline solid forms of Compound 1 and solid dosage forms thereof.

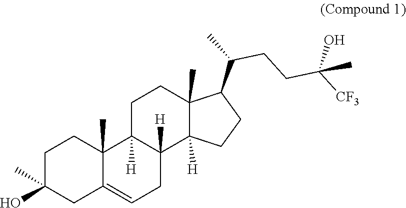

This application claims the benefit of and priority from U.S. Provisional Application No. 63/355,238, filed Jun. 24, 2022, the disclosure of which is incorporated by reference herein in its entirety.

### BACKGROUND

NMDA receptors are heteromeric complexes comprised of NR1, NR2, and/or NR3 subunits and possess distinct recognition sites for exogenous and endogenous ligands. These recognition sites include binding sites for glycine, and glutamate agonists and modulators. NMDA receptors are expressed in the peripheral tissues and the CNS, where they are involved in excitatory synaptic transmission. Activating these receptors contributes to synaptic plasticity in some circumstances and excitotoxicity in others. These receptors are ligand-gated ion channels that admit Ca²⁺ after binding of the glutamate and glycine and are fundamental to excitatory neurotransmission and normal CNS function. Positive modulators may be useful as therapeutic agents with potential clinical uses as cognitive enhancers and in the treatment of psychiatric disorders in which glutamatergic transmission is reduced or defective (see, e.g., Horak et al., J. of Neuroscience, 2004, 24 (46), 10318-10325).

Compound 1 disclosed herein is a positive allosteric modulator (PAM) of NMDA receptors. For a positive allosteric NMDA modulator to be a useful therapeutic, it must have an appropriate balance of various characteristic including affinity for the NMDA receptor, positive allosteric modulatory activity, duration of activity, oral bioavailability, tissue distribution, and stability (e.g., shelf life, formulatability, and crystallizability). The appropriate balance of such a combination of characteristics can lead to an NMDA receptor PAM having improved efficacy, safety, tolerability, patient compliance, manufacturing, etc. Therefore, large scale commercial preparations of a solid form of Compound 1 and its corresponding solid dosage forms which have the appropriate properties (including chemical stability, thermal stability, solubility, hygroscopicity, particle size, yield, impurity content during crystallization, drying properties, milling properties, and stability during tableting) present numerous challenges.

Accordingly, there is a current need for one or more solid forms of NMDA receptor PAMs (e.g., Compound 1) that have an appropriate balance of these characteristics and may be useful in preparing pharmaceutically acceptable solid dosage forms thereof.

### SUMMARY

Various polymorphic forms of Compound 1

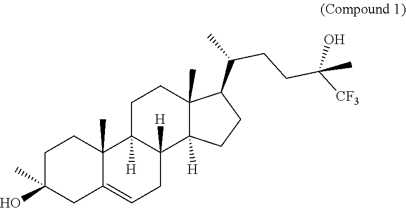

are disclosed herein, along with solid dosage forms comprising said forms of Compound 1 and methods of preparing and methods of using said forms of Compound 1.

In a first aspect, the disclosure provides a crystalline form of Compound 1:

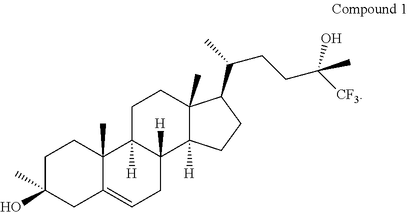

In some embodiments, the crystalline form of Compound 1 is selected from the group consisting of:
a) crystalline Form A, wherein Form A is characterized by an X-ray powder diffraction (XRPD) pattern comprising peaks at 15.3±0.2, 16.6±0.2, and 17.9±0.2 degrees 2θ;
b) crystalline Form B, wherein Form B is characterized by an XRPD pattern comprising peaks at 14.4±0.2, 16.8±0.2, and 18.5±0.2 degrees 2θ;
c) crystalline Form D, wherein Form D is characterized by an XRPD pattern comprising peaks at 15.2±0.2, 17.4±0.2 and 17.7±0.2 degrees 2θ;
d) crystalline Form E, wherein Form E is characterized by an XRPD pattern comprising peaks at 4.9±0.2, 12.0±0.2, 12.2±0.2, and 17.0±0.2 degrees 2θ;
e) crystalline Form F, wherein Form F is characterized by an XRPD pattern comprising peaks at 10.8±0.5, 17.1±0.5, 20.5±0.5, and 22.9±0.5 degrees 2θ; and
f) crystalline Form G, wherein Form G is characterized by an XRPD pattern comprising peaks at 9.1±0.5, 14.7±0.5, 16.0±0.5 and 17.2±0.5 degrees 2θ.

In some embodiments, the crystalline form of the Compound 1 is selected from the group consisting of:
a) crystalline Form A, wherein Form A is characterized by an X-ray powder diffraction (XRPD) pattern comprising peaks at 14.5±0.2, 15.3±0.2, 16.6±0.2, 17.9±0.2, and 20.1±0.2 degrees 2θ;
b) crystalline Form B, wherein Form B is characterized by an XRPD pattern comprising peaks at peaks at 10.9±0.2, 14.4±0.2, 16.8±0.2, 18.5±0.2 and 19.0±0.2 degrees 2θ;
c) crystalline Form D, wherein Form D is characterized by an XRPD pattern comprising peaks at 15.2±0.2, 16.4±0.2, 17.4±0.2, 17.7±0.2, 18.3±0.2, and 18.9±0.2 degrees 2θ;
d) crystalline Form E, wherein Form E is characterized by an XRPD pattern comprising peaks at 4.9±0.2, 12.0±0.2, 12.2±0.2, 17.0±0.2, and 21.4±0.2 degrees 2θ;
e) crystalline Form F, wherein Form F is characterized by an XRPD pattern comprising peaks at 7.6±0.5, 10.8±0.5, 13.6±0.5, 14.9±0.5, 15.4±0.5, 15.9±0.5, 17.1±0.5, 20.5±0.5, 22.8±0.5, and 22.9±0.5 degrees 2θ; and
f) crystalline Form G, wherein Form G is characterized by an XRPD pattern comprising peaks at 9.1±0.5, 12.0±0.5, 13.5±0.5, 14.7±0.5, 16.0±0.5, 17.2±0.5, and 17.7±0.5 degrees 2θ.

In some embodiments, the crystalline form of the Compound 1 is selected from the group consisting of:
a) crystalline Form A, wherein Form A is characterized by an X-ray powder diffraction (XRPD) pattern comprising peaks at 11.3±0.2, 14.5±0.2, 15.3±0.2, 16.6±0.2, 17.7±0.2, 17.9±0.2, and 20.1±0.2 degrees 2θ;
b) crystalline Form B, wherein Form B is characterized by an XRPD pattern comprising peaks at 10.9±0.2, 14.4±0.2, 15.4±0.2, 16.8±0.2, 18.5±0.2 and 19.0±0.2 degrees 2θ;
c) crystalline Form D, wherein Form D is characterized by an XRPD pattern comprising peaks at 9.1±0.2, 15.2±0.2, 16.4±0.2, 17.4±0.2, 17.7±0.2, 18.3±0.2, and 18.9±0.2 degrees 2θ;
d) crystalline Form E, wherein Form E is characterized by an XRPD pattern comprising peaks at 4.9±0.2, 8.5±0.2, 12.0±0.2, 12.2±0.2, 14.9±0.2, 15.8±0.2, 17.0±0.2, 18.2±0.2 and 21.4±0.2 degrees 2θ;
e) crystalline Form F, wherein Form F is characterized by an XRPD pattern comprising peaks at 7.6±0.5, 10.8±0.5, 13.6±0.5, 14.9±0.5, 15.4±0.5, 15.9±0.5, 17.1±0.5, 20.5±0.5, 22.8±0.5, and 22.9±0.5 degrees 2θ; and
f) crystalline Form G, wherein Form G is characterized by an XRPD pattern comprising peaks at 9.1±0.5, 12.0±0.5, 13.5±0.5, 14.7±0.5, 16.0±0.5, 17.2±0.5, and 17.7±0.5 degrees 2θ.

In some embodiments, the crystalline form is an anhydrate.

In some embodiments, the crystalline form is crystalline Form A characterized by an XRPD pattern comprising peaks at 15.3±0.2, 16.6±0.2, and 17.9±0.2 degrees 2θ. In some embodiments, the XRPD pattern of crystalline Form A further comprises at least one additional peak at a position selected from the group consisting of 11.3±0.2, 14.5±0.2, 17.7±0.2, and 20.1±0.2 degrees 2θ. In some embodiments, the crystalline form is crystalline Form A characterized by an XRPD pattern comprising peaks at 14.5±0.2, 15.3±0.2, 16.6±0.2, 17.9±0.2, and 20.1±0.2 degrees 2θ. In some embodiments, the crystalline form is crystalline Form A characterized by an XRPD pattern substantially as depicted in FIG. 1A.

In some embodiments, the crystalline Form A has a thermo-gravimetric analysis (TGA) thermogram substantially as depicted in FIG. 1B. In some embodiments, the crystalline Form A has a differential scanning calorimetry (DSC) thermogram substantially as depicted in FIG. 1B. In some embodiments, the DSC thermogram comprises an endothermic peak at about 203° C.

In some embodiments, the crystalline form is crystalline Form B characterized by an XRPD pattern comprising peaks at 14.4±0.2, 16.8±0.2, and 18.5±0.2 degrees 2θ. In some embodiments, the XRPD pattern of crystalline Form B further comprises at least one additional peak at a position selected from the group consisting of 10.9±0.2, 15.4±0.2, and 19.0±0.2 degrees 2θ. In some embodiments, the crystalline form is crystalline Form B characterized by an XRPD pattern comprising peaks at 10.9±0.2, 14.4±0.2, 16.8±0.2, 18.5±0.2 and 19.0±0.2 degrees 2θ. In some embodiments, the crystalline form is crystalline Form B characterized by an XRPD pattern substantially as depicted in FIG. 2A.

In some embodiments, the crystalline Form B has a TGA thermogram substantially as depicted in FIG. 2B. In some embodiments, the crystalline Form B has a DSC thermogram substantially as depicted in FIG. 2B. In some embodiments, the DSC thermogram comprises an endothermic peak at about 204° C.

In some embodiments, the crystalline form is a solvate. In some embodiments, the crystalline form is a hydrate. In some embodiments, the crystalline form is a monohydrate.

In some embodiments, the crystalline form is crystalline Form D characterized by an XRPD pattern comprising peaks at 15.2±0.2, 17.4±0.2 and 17.7±0.2 degrees 2θ. In some embodiments, the XRPD pattern of crystalline Form D further comprises at least one additional peak at a position selected from the group consisting of 9.1±0.2, 16.4±0.2, 18.3±0.2, and 18.9±0.2 degrees 2θ. In some embodiments, the crystalline form is crystalline Form D characterized by an XRPD pattern with characteristic peaks at 15.2±0.2, 16.4±0.2, 17.4±0.2, 17.7±0.2, 18.3±0.2, and 18.9±0.2 degrees 2θ. In some embodiments, the crystalline form is crystalline Form D characterized by an XRPD pattern substantially as depicted in FIG. 3A.

In some embodiments, the crystalline Form D has a TGA thermogram substantially as depicted in FIG. 3B. In some embodiments, the crystalline Form D has a differential scanning calorimetry (DSC) thermogram substantially as in FIG. 3B. In some embodiments, the DSC thermogram comprises an endothermic peak at about 204° C. In some embodiments, the DSC thermogram comprises an endothermic peak at about 127° C.

In some embodiments, the crystalline form is a n-BuOH solvate. In some embodiments, the crystalline form is crystalline Form E characterized by an XRPD pattern comprising peaks at 4.9±0.2, 12.0±0.2, 12.2±0.2, and 17.0±0.2 degrees 2θ. In some embodiments, the XRPD pattern of crystalline Form E further comprises at least one additional peak at a position selected from the group consisting of at 8.5±0.2, 14.9±0.2, 15.8±0.2, 18.2±0.2 and 21.4±0.2 degrees 2θ. In some embodiments, the crystalline form is crystalline Form E characterized by having an XRPD pattern with characteristic peaks at 4.9±0.2, 12.0±0.2, 12.2±0.2, 17.0±0.2, and 21.4±0.2 degrees 2θ. In some embodiments, the crystalline form is crystalline Form E characterized by an XRPD pattern substantially as depicted in FIG. 4A.

In some embodiments, the crystalline Form E has a TGA thermogram substantially as depicted in FIG. 4B. In some embodiments, the crystalline Form E has a differential scanning calorimetry (DSC) thermogram substantially as depicted in FIG. 4B. In some embodiments, the thermogram comprises an endothermic peak at about 203° C. In some embodiments, the DSC thermogram comprises an endothermic peak at about 87° C.

In some embodiments, the crystalline form is crystalline Form F characterized by an XRPD pattern comprising peaks at 10.8±0.5, 17.1±0.5, 20.5±0.5, and 22.9±0.5 degrees 2θ. In some embodiments, the XRPD pattern of crystalline Form F further comprises at least one additional peak at a position selected from the group consisting of 7.6±0.5, 13.6±0.5, 14.9±0.5, 15.4±0.5, 15.9±0.5 and 22.8±0.5 degrees 2θ. In some embodiments, the crystalline form is crystalline Form F characterized by an XRPD pattern comprising peaks at 7.6±0.5, 10.8±0.5, 13.6±0.5, 14.9±0.5, 15.4±0.5, 15.9±0.5, 17.1±0.5, 20.5±0.5, 22.8±0.5, and 22.9±0.5 degrees 2θ. In some embodiments, the crystalline form is crystalline Form F characterized by an XRPD pattern substantially as depicted in FIG. 5A.

In some embodiments, the crystalline form is crystalline Form G characterized by an XRPD pattern comprising peaks at 9.1±0.5, 14.7±0.5, 16.0±0.5 and 17.2±0.5 degrees 2θ. In some embodiments, the XRPD pattern of crystalline Form G further comprises at least one additional peak at a position selected from the group consisting of 12.0±0.5, 13.5±0.5 and 17.7±0.5 degrees 2θ. In some embodiments, the crystalline form is crystalline Form G characterized by an XRPD pattern comprising peaks at 9.1±0.5, 12.0±0.5, 13.5±0.5, 14.7±0.5, 16.0±0.5, 17.2±0.5, and 17.7±0.5 degrees 2θ. In some embodiments, the crystalline form is crystalline Form G characterized by an XRPD pattern substantially as depicted in FIG. 5A.

In some embodiments, the crystalline form is selected from the group consisting of:
a) crystalline Form A characterized by an XRPD pattern substantially as depicted in FIG. 1A;
b) crystalline Form B characterized by an XRPD pattern substantially as depicted in FIG. 2A;
c) crystalline Form D characterized by an XRPD pattern substantially as depicted in FIG. 3A;
d) crystalline Form E characterized by an XRPD pattern substantially as depicted in FIG. 4A;
e) crystalline Form F characterized by an XRPD pattern substantially as depicted in FIG. 5A; and
f) crystalline Form G characterized by an XRPD pattern substantially as depicted in FIG. 5A.

In a second aspect, the disclosure provides a solid dosage form comprising a crystalline form of Compound 1 as disclosed herein and at least one pharmaceutically acceptable carrier.

In a third aspect, the disclosure provides a method of treating a CNS-related condition in a subject comprising administering to the subject an effective amount of a crystalline form of Compound 1 as disclosed herein, or solid dosage form comprising a crystalline form of Compound 1. In some embodiments, the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

In a fourth aspect, the disclosure provides a method of inducing sedation or anesthesia in a subject, comprising administering to the subject an effective amount of a crystalline form of Compound 1 as disclosed herein, or a solid dosage form comprising a crystalline form of Compound 1 as disclosed herein.

In a fifth aspect, the disclosure provides a method of making a crystalline form of Compound 1. In some embodiments, the method of making a crystalline form of Compound 1 comprises the steps of: a) dissolving Compound 1 in at least one solvent to form a solution; b) adding an anti-solvent until a precipitate forms; and c) isolating the precipitate. In some embodiments, the solvent is selected from the group consisting of THF, 1,4-dioxane and MEK. In some embodiments, the anti-solvent is selected from the group consisting of H₂O and ACN.

In some embodiments, the method of making a crystalline form of Compound 1 comprises the steps of: a) adding Compound 1 to a solvent to form a mixture; b) aging the mixture at room temperature to precipitate solids; and c) isolating the solids. In some embodiments, the solvent is selected from the group consisting of DME, Acetone, nBuOH, EtOAc, IPAc, BuOAc, diethyl ether, and ACN.

In some embodiments, the method of making a crystalline form of Compound 1 comprises the steps of: a) adding Compound 1 to a solvent to form a mixture; b) aging the mixture at about 40° C. to precipitate solids; and c) isolating the solids. In some embodiments, the solvent is selected from the group consisting of IPAc, BuOAc, ACN, and n-heptane.

In some embodiments, the method of making a crystalline form of Compound 1 comprises the steps of: a) adding Compound 1 to a volatile solvent to form a mixture; b) aging the mixture at room temperature to allow the volatile solvent vapor to interact with Compound 1 and to precipitate solids; and c) isolating the solids. In some embodiments, the solvent is selected from the group consisting of DME, MEK, diethyl ether and t-BuOH.

In some embodiments, the method of making a crystalline form of Compound 1 comprises the steps of: a) adding Compound 1 to solvent to form a solution; b) adding the solution to an anti-solvent; c) aging the mixture at room temperature to precipitate solids; and c) isolating the solids. In some embodiments, the solvent is BuOH. In some embodiments, the anti-solvent is n-Heptane.

In some embodiments, the method of making a crystalline form of Compound 1 comprises the steps of: a) adding Compound 1 to solvent to form a mixture; b) filtering the mixture to provide a filtrate; c) aging the filtrate at room temperature; d) allowing solids to precipitate; and e) isolating the solids. In some embodiments, the solvent is selected from the group consisting of IPAc, and t-BuOH.

In some embodiments, the method of making a crystalline form of Compound 1 comprises the steps of: a) adding Compound 1 to solvent to form a mixture; b) heating the mixture to about 50° C.; c) filtering the mixture to provide a filtrate; c) cooling the filtrate to about −20° C.; d) allowing solids to precipitate solids; and e) isolating the solids. In some embodiments, the solvent is acetone.

In some embodiments, the method of making a crystalline form of Compound 1 comprises the steps of: a) heating Compound 1 to about 205° C.; b) cooling the heated Compound 1 to about 25° C. to provide solids; and c) isolating the solids. In some embodiments, the cooling in step b) is a rate selected from the group consisting of about 2° C./min, about 10° C./min and about 50° C./min.

In some embodiments, the method of making a crystalline form of Compound 1 comprises the steps of: a) adding Compound 1 to solvent to form a mixture; b) aging the mixture at about 90° C. to precipitate solids; and d) isolating the solids. In some embodiments, the solvent is n-heptane.

In some embodiments, the method of making a crystalline form of Compound 1 comprises the steps of: a) adding Compound 1 to solvent to form a mixture; b) grinding the mixture to provide solids; and d) isolating the solids. In some embodiments, the solvent is acetone/MIBK.

In some embodiments, the method of making a crystalline form of Compound 1 comprises the steps of: a) adding Form E of Compound 1 to a first solvent to form a mixture; b) concentrating the mixture by distillation at atmospheric pressure; c) cooling the mixture to about 80-85° C.; d) adding a second solvent to the mixture and maintaining the temperature of the mixture at about 80-85° C. for at least 1 hour; e) concentrating the mixture by distillation at atmospheric pressure; f) cooling the mixture to about 20° C. to precipitate solids; and g) isolating the solids. In some embodiments, the first solvent is heptane. In some embodiments, the second solvent is n-BuOH.

In some embodiments, the disclosure provides a method of making a crystalline form of Compound 1 comprising the steps of: a) adding Form E of Compound 1 to a solvent to form a mixture; b) heating the mixture to about 50° C.; c) adding antisolvent; d) adding Form X of Compound 1 seeds and aging the mixture at about 50° C.; e) cooling the mixture to about 20° C. over at least 3 hours and holding the mixture at 20° C. (wet milling optional) to precipitate solids; and f) isolating the solids. In some embodiments, the solvent is acetone. In some embodiments, the anti-solvent is acetone/water. In some embodiments, the anti-solvent is acetone/water (1:1 v/v).

In some embodiments, the disclosure provides a method of making a crystalline form of Compound 1 comprising the steps of: a) adding crude Compound 1 to a solvent to form a mixture and heating to about 80° C.; b) concentrating the mixture via distillation; c) adding solvent to the mixture; d) adjusting the temperature of the mixture to about 80° C.; e) adding Form E of Compound 1 seeds; f) cooling the mixture to about 15-25° C.; g) aging the mixture at about 15-25° C. for about 4-18 hours to precipitate solids; and h) isolating the solids. In some embodiments, the solvent is n-BuOH.

### BRIEF DESCRIPTION OF THE DRAWINGS

FIG. 1A depicts an exemplary XRPD pattern for Form A.

FIG. 1B depicts exemplary TGA (upper) and DSC (lower) thermogram profiles for Form A.

FIG. 1C depicts an exemplary hot stage image for Form A.

FIG. 1D depicts an exemplary DVS plot for Form A.

FIG. 1E depicts an exemplary XRPD overlay of Form A before and after DVS test.

FIG. 2A depicts an exemplary XRPD pattern for Form B.

FIG. 2B depicts exemplary TGA (upper) and DSC (lower) thermogram profiles for Form B.

FIG. 3A depicts an exemplary XRPD pattern for Form D.

FIG. 3B depicts exemplary TGA (upper) and DSC (lower) thermogram profiles for Form D.

FIG. 3C depicts an exemplary XRPD overlay of Form D before and after heating.

FIG. 4A depicts an exemplary XRPD pattern for Form E.

FIG. 4B depicts exemplary TGA (upper) and DSC (lower) thermogram profiles for Form E.

FIG. 4C depicts an exemplary XRPD overlay of Form E before and after heating.

FIG. 4D depicts an exemplary ¹H-NMR spectrum for Form E.

FIG. 5A depicts an exemplary XRPD pattern for Form F: T XRPD patterns of Compound 1 samples: Form X: reference; Form A: Compound 1 ref. Form F: #1 oven dried at 40° C.); Form G: #2 ref, heated at 90° C.

FIG. 5B depicts an exemplary TGA thermogram for Form F from slurry conversion post drying in vacuum oven at 40° C.

FIG. 5C depicts an exemplary DSC thermogram for Form F from slurry conversion post drying in vacuum oven at 40° C.

### DETAILED DESCRIPTION

### General Definitions

The term “herein” means the entire application.

Unless otherwise defined herein, scientific and technical terms used in this application shall have the meanings that are commonly understood by those of ordinary skill in the art to which this disclosure belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments of the disclosure, unless explicitly disclaimed or improper. Combination of embodiments are not limited to those specific combinations claimed via the multiple dependent claims. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range and including the endpoints, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments, and does not pose a limitation on the scope of the claims unless otherwise stated. No language in the specification should be construed as indicating any non-claimed element as essential.

All of the publications, patents and published patent applications referred to in this application are specifically incorporated by reference herein. In case of conflict, the present specification, including its specific definitions, will control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The terms “active ingredient,” “active agent” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient,” “active agent” and “active substance” may be an optically active isomer of a compound described herein.

The terms “drug,” and “therapeutic agent,” refer to a compound, or a pharmaceutical composition thereof, which is administered to a subject for treating, preventing, or ameliorating one or more symptoms of a condition, disorder, or disease.

The term “polymorph” as used herein refers to a crystalline form of a compound or a salt, hydrate, or solvate thereof, in a particular crystal packing arrangement. All polymorphs have the same elemental composition. The term “polymorph” includes pseudopolymorphs, which are typically different solvates of a material, and thus their properties differ from one another. Thus, each distinct polymorph and pseudopolymorph disclosed herein is considered to be a distinct single crystalline form herein.

The term “crystalline,” as used herein, refers to a solid state form which consists of orderly arrangement of structural units. Different crystalline forms of the same compound, or a salt, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility. See, e.g., Remington's Pharmaceutical Sciences, 18th ed., Mack Publishing, Easton Pa., 173 (1990); The United States Pharmacopeia, 23.sup.rd ed., 1843-1844 (1995) (incorporated herein by reference). As used herein, the terms “crystalline form,” “single crystalline form,” “crystalline solid form,” and “polymorph” are used interchangeably; the terms distinguish between crystals that have different properties (e.g., different XRPD patterns and/or different DSC scan results).

The term “substantially pure” relates to the composition of a specific crystalline solid form of Compound 1 that may be at least a particular weight percent free of impurities and/or other solid forms of Compound 1. Particular weight percentages are 70%, 75%, 80%, 85%, 90%, 95%, 99%, or any percentage between 70% and 100%. In some embodiments, Compound 1 can be a substantially pure sample of any of the crystalline solid forms described herein. In some embodiments, Compound 1 can be substantially pure Form A. In some embodiments, Compound 1 can be substantially pure Form B. In some embodiments, Compound 1 can be substantially pure Form D. In some embodiments, Compound 1 can be substantially pure Form E. In some embodiments, Compound 1 can be substantially pure Form F. In some embodiments, Compound 1 can be substantially pure Form G.

Crystalline forms are commonly characterized by X-ray powder diffraction (XRPD). An XRPD pattern of reflections (peaks, typically expressed in degrees 2-theta) is generally considered a fingerprint of a particular crystalline form. The relative intensities of the XRPD peaks may vary depending on, inter alia, the sample preparation technique, crystal size distribution, filters, the sample mounting procedure, and the particular instrument employed. In some instances, more (i.e., new peaks) or fewer (i.e., peaks may disappear) peaks may be present in the XRPD pattern, depending on the type of instrument or the settings. In some instances, any particular peak in an XRPD pattern may appear as a singlet, doublet, triplet, quartet, or multiplet, depending on the type of instrument or the settings, the sensitivity of the instrument, measuring conditions, and/or purity of the crystalline form. In some instances, any particular peak in an XRPD may appear in a symmetric shape or in an asymmetric shape, e.g., having a shoulder. Moreover, instrument variation and other factors can affect the 2 -theta values. A skilled artisan understanding these variations is capable of identifying or ascertaining the defining features or characteristics of a particular crystal form using XRPD, as well as using other known physicochemical techniques.

The term “characteristic peaks” when referring to the peaks in an XRPD pattern of a crystalline form of Compound 1 refers to a collection of certain peaks whose values of 2θ across a range of 0°-40° are, as a whole, uniquely assigned to one of the crystalline forms of Compound 1.

The term “amorphous” as applied to a compound refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”).

The term “anhydrate” or “anhydrous” are used interchangeably and as applied to a compound refers to a solid state wherein the compound contains no structural water or other solvent molecule within the crystal lattice.

The term “solvate” refers to a compound provided herein or a salt thereof, which further includes a stoichiometric or non-stoichiometric amount of solvent bound by non-covalent intermolecular forces (e.g., hydrogen bonding). Where the solvent is water, the solvate is a hydrate. Where the solvent includes ethanol, the compound can be an ethanol solvate.

The term “stable” in the context of a polymorphic form disclosed herein refers to the stability of the polymorphic form relative to heat and/or humidity.

A crystalline form of Compound 1 described herein, e.g., Form A, can melt at a specific temperature or across a range of temperatures. Such a specific temperature or range of temperatures can be represented by the onset temperature (Tₒₙₛₑₜ) of the melting endotherm in the crystalline form's DSC trace. In some embodiments, at such an onset temperature, a sample of a crystalline form of Compound 1 melts and undergoes a concurrently occurring side-process, e.g., recrystallization or chemical decomposition. In some embodiments, at such an onset temperature, a crystalline form of Compound 1 melts in the absence of other concurrently occurring processes.

The present disclosure provides forms of Compound 1, which have the appropriate properties for preparing solid dosage forms. Such properties include chemical stability, thermal stability, solubility, hygroscopicity, particle size, yield, impurity content during crystallization, drying properties, milling properties, and stability during tableting.

The present disclosure relates to a solid form of Compound 1

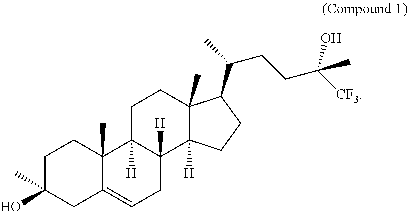

In some embodiments, the solid form is a crystalline form of Compound 1. In some embodiments, the crystalline form is anhydrous. In some embodiments, the crystalline form is a hydrate. In some embodiments, the crystalline form is a solvate. Exemplary individual crystalline forms of the present disclosure are Forms A, B, D, E, F and G.

Compound 1 and its chemical synthesis are disclosed in in U.S. Pat. No. 10,227,375/PCT Application Publication No. WO2014160480.

In some embodiments, the crystalline structures of this disclosure can be identified by having one or more characteristics peaks in an XRPD spectrum, as disclosed herein.

In some embodiments, the crystalline structures of this disclosure can be identified by having one or more characteristics endothermic peaks in a differential scanning calorimetry (DSC) thermogram, as disclosed herein.

In some embodiments, the crystalline structures of this disclosure can be identified by having one or more characteristics endothermic peaks in a thermo-gravimetric analysis (TGA) thermogram, as disclosed herein.

In some embodiments, the crystalline structures of this disclosure can be identified by having one or more characteristics endothermic peaks in a differential scanning calorimetry (DSC) thermogram or thermo-gravimetric analysis (TGA) thermogram, as disclosed herein.

In some embodiments, crystalline structures of this disclosure can be identified by having one or more characteristics peaks in an XRPD spectrum in combination with having one or more characteristics endothermic peaks in a differential scanning calorimetry thermogram and/or a thermo-gravimetric analysis (TGA) thermogram.

In some embodiments, the crystalline form of Compound 1 is selected from the group consisting of:
a) crystalline Form A, wherein Form A is characterized by an X-ray powder diffraction (XRPD) pattern comprising peaks at 15.3±0.2, 16.6±0.2, and 17.9±0.2 degrees 2θ;
b) crystalline Form B, wherein Form B is characterized by an XRPD pattern comprising peaks at 14.4±0.2, 16.8±0.2, and 18.5±0.2 degrees 2θ;
c) crystalline Form D, wherein Form D is characterized by an XRPD pattern comprising peaks at 15.2±0.2, 17.4±0.2 and 17.7±0.2 degrees 2θ;
d) crystalline Form E, wherein Form E is characterized by an XRPD pattern comprising peaks at 4.9±0.2, 12.0±0.2, 12.2±0.2, and 17.0±0.2 degrees 2θ;
e) crystalline Form F, wherein Form F is characterized by an XRPD pattern comprising peaks at 10.8±0.5, 17.1±0.5, 20.5±0.5, and 22.9±0.5 degrees 2θ; and
f) crystalline Form G, wherein Form G is characterized by an XRPD pattern comprising peaks at 9.1±0.5, 14.7±0.5, 16.0±0.5 and 17.2±0.5 degrees 2θ.

In some embodiments, the crystalline form of Compound 1 is selected from the group consisting of:
a) crystalline Form A, wherein Form A is characterized by an X-ray powder diffraction (XRPD) pattern comprising peaks at 14.5±0.2, 15.3±0.2, 16.6±0.2, 17.9±0.2, and 20.1±0.2 degrees 2θ;
b) crystalline Form B, wherein Form B is characterized by an XRPD pattern comprising peaks at peaks at 10.9±0.2, 14.4±0.2, 16.8±0.2, 18.5±0.2 and 19.0±0.2 degrees 2θ;
c) crystalline Form D, wherein Form D is characterized by an XRPD pattern comprising peaks at 15.2±0.2, 16.4±0.2, 17.4±0.2, 17.7±0.2, 18.3±0.2, and 18.9±0.2 degrees 2θ;
d) crystalline Form E, wherein Form E is characterized by an XRPD pattern comprising peaks at 4.9±0.2, 12.0±0.2, 12.2±0.2, 17.0±0.2, and 21.4±0.2 degrees 2θ;
e) crystalline Form F, wherein Form F is characterized by an XRPD pattern comprising peaks at 7.6±0.5, 10.8±0.5, 13.6±0.5, 14.9±0.5, 15.4±0.5, 15.9±0.5, 17.1±0.5, 20.5±0.5, 22.8±0.5, and 22.9±0.5 degrees 2θ; and
f) crystalline Form G, wherein Form G is characterized by an XRPD pattern comprising peaks at 9.1±0.5, 12.0±0.5, 13.5±0.5, 14.7±0.5, 16.0±0.5, 17.2±0.5, and 17.7±0.5 degrees 2θ.

In some embodiments, the crystalline form of Compound 1 is selected from the group consisting of:
a) crystalline Form A, wherein Form A is characterized by an X-ray powder diffraction (XRPD) pattern comprising peaks at 11.3±0.2, 14.5±0.2, 15.3±0.2, 16.6±0.2, 17.7±0.2, 17.9±0.2, and 20.1±0.2 degrees 2θ;
b) crystalline Form B, wherein Form B is characterized by an XRPD pattern comprising peaks at 10.9±0.2, 14.4±0.2, 15.4±0.2, 16.8±0.2, 18.5±0.2 and 19.0±0.2 degrees 2θ;
c) crystalline Form D, wherein Form D is characterized by an XRPD pattern comprising peaks at 9.1±0.2, 15.2±0.2, 16.4±0.2, 17.4±0.2, 17.7±0.2, 18.3±0.2, and 18.9±0.2 degrees 2θ;
d) crystalline Form E, wherein Form E is characterized by an XRPD pattern comprising peaks at 4.9±0.2, 8.5±0.2, 12.0±0.2, 12.2±0.2, 14.9±0.2, 15.8±0.2, 17.0±0.2, 18.2±0.2 and 21.4±0.2 degrees 2θ;
e) crystalline Form F, wherein Form F is characterized by an XRPD pattern comprising peaks at 7.6±0.5, 10.8±0.5, 13.6±0.5, 14.9±0.5, 15.4±0.5, 15.9±0.5, 17.1±0.5, 20.5±0.5, 22.8±0.5, and 22.9±0.5 degrees 2θ; and
f) crystalline Form G, wherein Form G is characterized by an XRPD pattern comprising peaks at 9.1±0.5, 12.0±0.5, 13.5±0.5, 14.7±0.5, 16.0±0.5, 17.2±0.5, and 17.7±0.5 degrees 2θ.

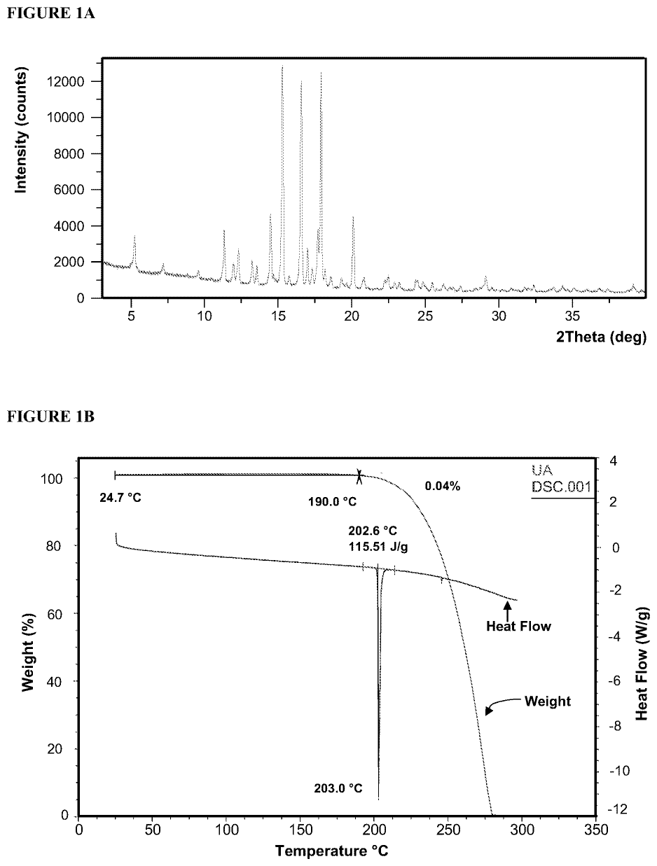

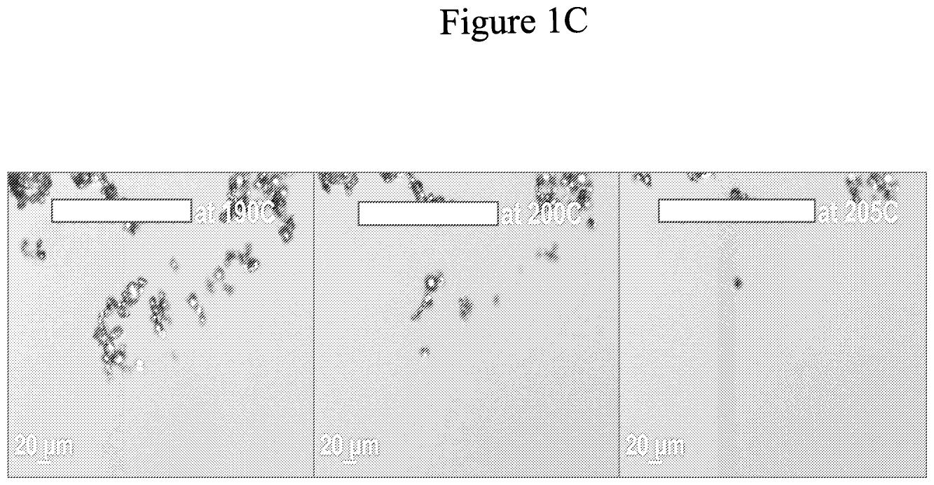

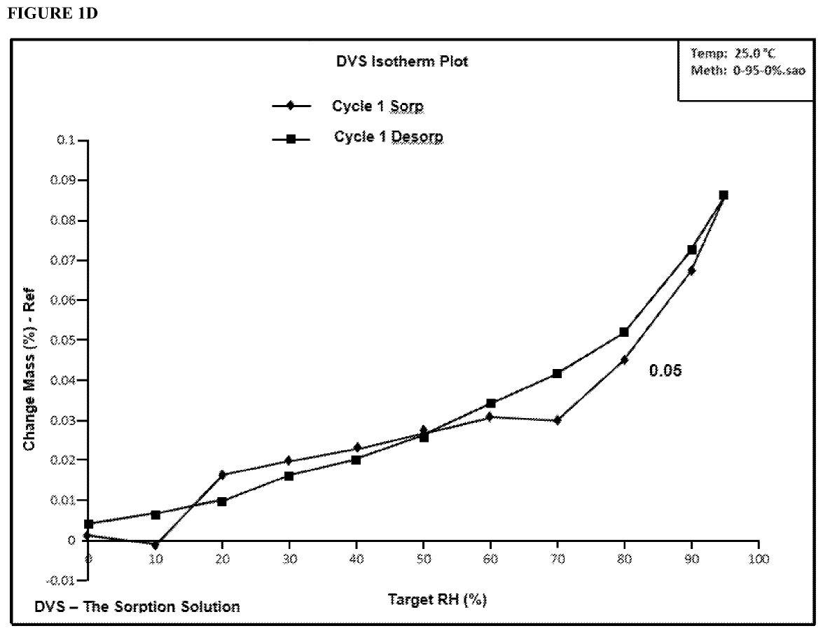

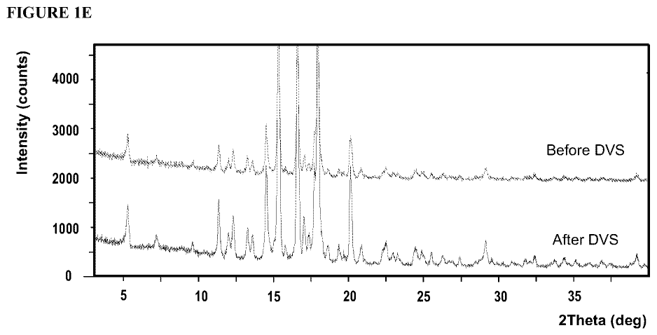

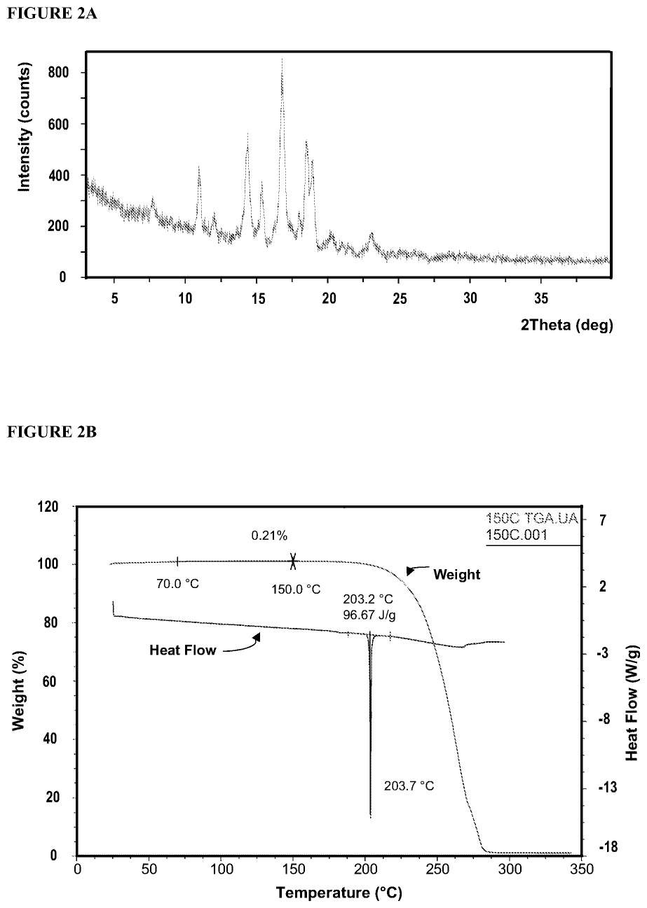

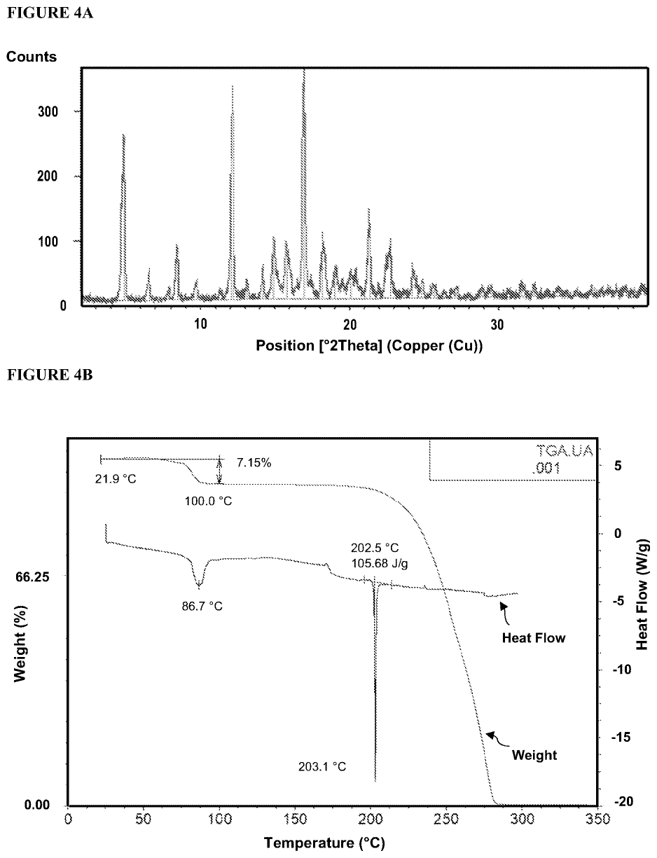

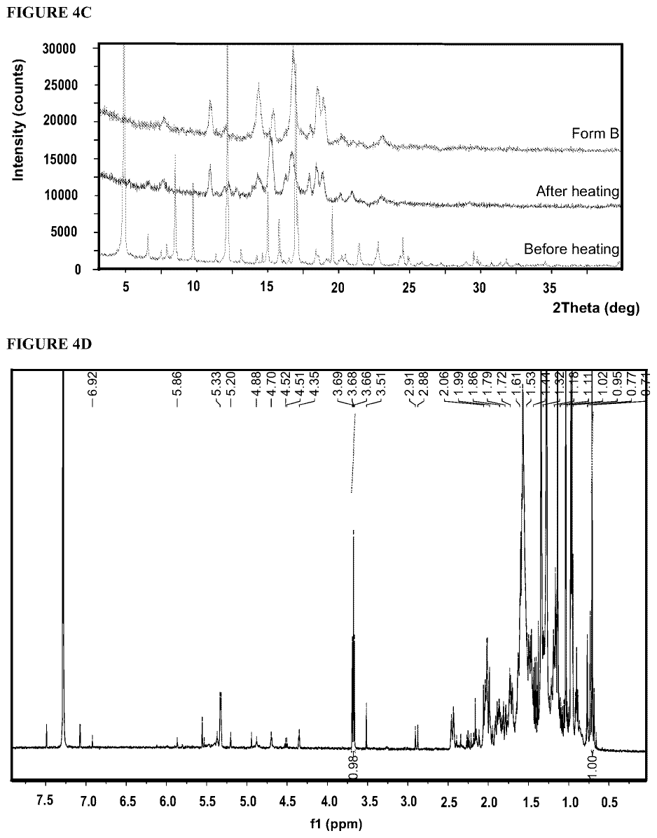

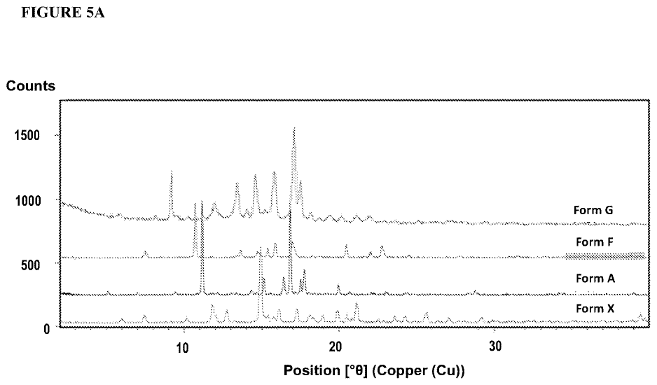

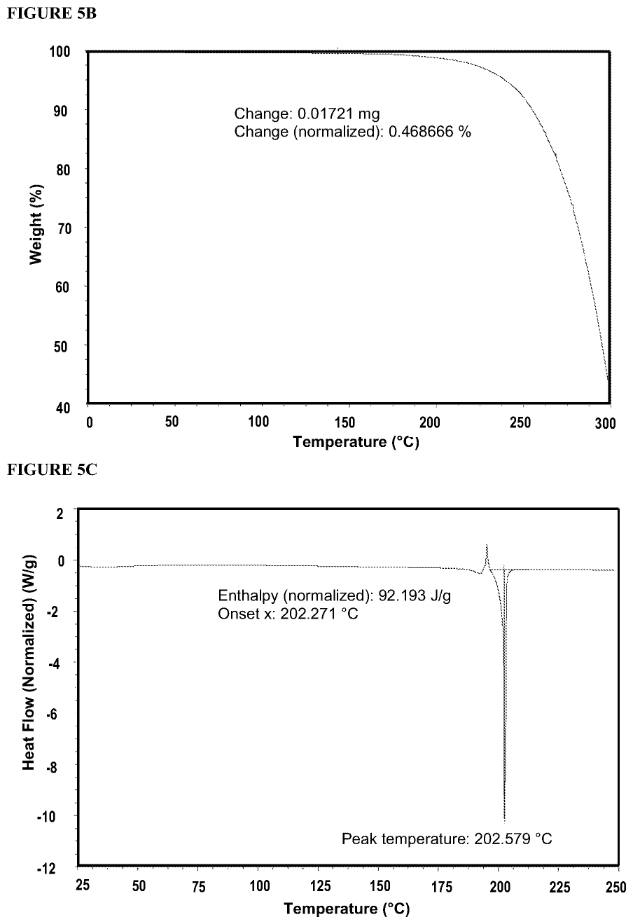

In some embodiments, the crystalline form of Compound 1 is selected from the group consisting of:
a) crystalline Form A, wherein Form A is characterized by an X-ray powder diffraction (XRPD) pattern substantially as shown in FIG. 1A;
b) crystalline Form B, wherein Form B is characterized by an XRPD pattern substantially as shown in FIG. 2A;
c) crystalline Form D, wherein Form D is characterized by an XRPD pattern 3A;
d) crystalline Form E, wherein Form E is characterized by an XRPD pattern substantially as shown in FIG. 4A;
e) crystalline Form F, wherein Form F is characterized by an XRPD pattern substantially as shown in FIG. 5A; and
f) crystalline Form G, wherein Form G is characterized by an XRPD pattern substantially as shown in FIG. 5A.

In some embodiments, the crystalline form of Compound 1 is an anhydrate. In some embodiments, the crystalline form of Compound 1 is a solvate. In some embodiments, the crystalline form of Compound 1 is a hydrate. In some embodiments, the crystalline form of Compound 1 is a monohydrate. In some embodiments, the crystalline form of Compound 1 is an n-butanol solvate.

### A. Crystalline Form A

In some embodiments, the present disclosure relates to crystalline Form A of Compound 1. In some embodiments, the anhydrous crystalline form of Compound 1 is Form A. In some embodiments, Form A has one or more characteristics selected from the group consisting of:
a) an XRPD pattern comprising peaks at 15.3±0.2, 16.6±0.2, and 17.9±0.2 degrees 2θ;
b) an XRPD pattern substantially as shown in FIG. 1A;
c) a DSC thermogram profile comprising an endothermic peak at about 203° C.;
d) a DSC thermogram profile substantially as shown in FIG. 1B;
e) a TGA profile exhibiting a loss of at least about 0.04 wt % between about 25° C. and about 190° C.; and
f) a TGA profile substantially as shown in FIG. 1B.

In some embodiments, Form A is characterized by an XRPD pattern comprising peaks at 15.3±0.2, 16.6±0.2, and 17.9±0.2 degrees 2θ. In some embodiments, Form A is characterized by an XRPD pattern comprising peaks at 15.3±0.2, 16.6±0.2, and 17.9±0.2 degrees 2θ, and further comprising at least one additional peak at a position selected from the group consisting of 11.3±0.2, 14.5±0.2, 17.7±0.2, and 20.1±0.2 degrees 2θ. In some embodiments, Form A is characterized by an XRPD pattern comprising peaks at 14.5±0.2, 15.3±0.2, 16.6±0.2, 17.9±0.2, and 20.1±0.2 degrees 2θ. In some embodiments, Form A is characterized by an XRPD pattern comprising peaks at 11.3±0.2, 14.5±0.2, 15.3±0.2, 16.6±0.2, 17.7±0.2, 17.9±0.2, and 20.1±0.2 degrees 2θ. In some embodiments, crystalline Form A is characterized by an XRPD pattern substantially as depicted in FIG. 1A.

In some embodiment, Form A is characterized by a thermo-gravimetric analysis (TGA) thermogram exhibiting that Form A loses at least about 0.04 wt % between about 25° C. and about 190° C. In some embodiment, Form A is characterized by a thermo-gravimetric analysis (TGA) thermogram exhibiting that Form A loses about 0.04 wt % between about 25° C. and about 190° C. In some embodiments, Form A is characterized by a thermo-gravimetric analysis (TGA) thermogram substantially as depicted in FIG. 1B.

In some embodiments, Form A is characterized by a differential scanning calorimetry (DSC) thermogram substantially as depicted in FIG. 1B. In some embodiments, Form A is characterized by a DSC thermogram comprising an endothermic peak at about 203° C. In some embodiments, Form A is characterized by a DSC thermogram comprising a melting endothermic peak at about 203° C.

In some embodiments, Form A is substantially free of other polymorphic forms. In some embodiments, Form A has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form A has a polymorphic purity of at least 90%. In some embodiments, Form A has a polymorphic purity of at least 92%. In some embodiments, Form A has a polymorphic purity of at least 94%. In some embodiments, Form A has a polymorphic purity of at least 96%. In some embodiments, Form A has a polymorphic purity of at least 98%. In some embodiments, Form A has a polymorphic purity of at least 99%.

### B. Crystalline Form B

In some embodiments, the present disclosure relates to crystalline Form B of Compound 1. In some embodiments, the anhydrous crystalline form of Compound 1 is Form B. In some embodiments, Form B has one or more characteristics selected from the group consisting of:
a) an XRPD pattern comprising peaks at 14.4±0.2, 16.8±0.2, and 18.5±0.2 degrees 2θ;
b) an XRPD pattern substantially as shown in FIG. 2A;
c) a DSC thermogram profile comprising an endothermic peak at about 204° C.;
d) a DSC thermogram profile substantially as shown in FIG. 2B;
e) a TGA profile exhibiting a loss of at least about 0.2 wt % between about 70° C. and about 150° C.; and
f) a TGA profile substantially as shown in FIG. 2B.

In some embodiments, Form B is characterized by an XRPD pattern comprising peaks at 14.4±0.2, 16.8±0.2, and 18.5±0.2 degrees 2θ. In some embodiments, Form B is characterized by an XRPD pattern comprising peaks at 14.4±0.2, 16.8±0.2, and 18.5±0.2 degrees 2θ, and further comprising at least one additional peak at a position selected from the group consisting of 10.9±0.2, 15.4±0.2, and 19.0±0.2 degrees 2θ. In some embodiments, Form B is characterized by an XRPD pattern comprising peaks at 10.9±0.2, 14.4±0.2, 16.8±0.2, 18.5±0.2 and 19.0±0.2 degrees 2θ. In some embodiments, Form B is characterized by an XRPD pattern comprising peaks at 10.9±0.2, 14.4±0.2, 15.4±0.2, 16.8±0.2, 18.5±0.2 and 19.0±0.2 degrees 2θ. In some embodiments, crystalline Form B is characterized by an XRPD pattern substantially as depicted in FIG. 2A.

In some embodiment, Form B is characterized by a thermo-gravimetric analysis (TGA) thermogram exhibiting that Form B loses at least about 0.2 wt % between about 70° C. and about 150° C. In some embodiments, Form B is characterized by a thermo-gravimetric analysis (TGA) thermogram substantially as depicted in FIG. 2B.

In some embodiments, Form B is characterized by a differential scanning calorimetry (DSC) thermogram substantially as depicted in FIG. 2B. In some embodiments, Form B is characterized by a DSC thermogram comprising an endothermic peak at about 204° C. In some embodiments, Form B is characterized by a DSC thermogram comprising a melting endothermic peak at about 204° C.

In some embodiments, Form B is substantially free of other polymorphic forms. In some embodiment, Form B has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form B has a polymorphic purity of at least 90%. In some embodiments, Form B has a polymorphic purity of at least 92%. In some embodiments, Form B has a polymorphic purity of at least 94%. In some embodiments, Form B has a polymorphic purity of at least 96%. In some embodiments, Form B has a polymorphic purity of at least 98%. In some embodiments, Form B has a polymorphic purity of at least 99%.

### C. Crystalline Form F

In some embodiments, the present disclosure relates to crystalline Form F of Compound 1. In some embodiments, the anhydrous crystalline form of Compound 1 is Form F. In some embodiments Form F has one or more characteristics selected from the group consisting of:
a) an XRPD pattern comprising peaks at 10.8±0.5, 17.1±0.5, 20.5±0.5, and 22.9±0.5 degrees 2θ;
b) an XRPD pattern substantially as shown in FIG. 5A;
c) a DSC thermogram profile comprising an endothermic peak at about 203° C.;
d) a DSC thermogram profile substantially as shown in FIG. 5B; and
e) a TGA profile substantially as shown in FIG. 5C.

In some embodiments, Form F is characterized by an XRPD pattern comprising peaks at 10.8±0.5, 17.1±0.5, 20.5±0.5, and 22.9±0.5 degrees 2θ. In some embodiments, Form F is characterized by an XRPD pattern comprising peaks at 10.8±0.5, 17.1±0.5, 20.5±0.5, and 22.9±0.5 degrees 2θ, and further comprising at least one additional peak at a position selected from the group consisting of 7.6±0.5, 13.6±0.5, 14.9±0.5, 15.4±0.5, 15.9±0.5 and 22.8±0.5 degrees 2θ. In some embodiments, Form F is characterized by an XRPD pattern comprising peaks at 7.6±0.5, 10.8±0.5, 13.6±0.5, 14.9±0.5, 15.4±0.5, 15.9±0.5, 17.1±0.5, 20.5±0.5, 22.8±0.5, and 22.9±0.5 degrees 2θ. In some embodiments, crystalline Form F is characterized by an XRPD pattern substantially as depicted in FIG. 5A.

In some embodiments, Form F is substantially free of other polymorphic forms. In some embodiments, Form F has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form F has a polymorphic purity of at least 90%. In some embodiments, Form F has a polymorphic purity of at least 92%. In some embodiments, Form F has a polymorphic purity of at least 94%. In some embodiments, Form F has a polymorphic purity of at least 96%. In some embodiments, Form F has a polymorphic purity of at least 98%. In some embodiments, Form F has a polymorphic purity of at least 99%.

### D. Crystalline Form G

In some embodiments, the present disclosure relates to crystalline Form G of Compound 1. In some embodiments, the anhydrous crystalline form of Compound 1 is Form G. In some embodiments, Form G has one or more characteristics selected from the group consisting of:
a) an XRPD pattern comprising peaks at 9.1±0.5, 14.7±0.5, 16.0±0.5 and 17.2±0.5 degrees 2θ; and
b) an XRPD pattern substantially as shown in FIG. 5A.

In some embodiments, Form G is characterized by an XRPD pattern comprising peaks at 9.1±0.5, 14.7±0.5, 16.0±0.5 and 17.2±0.5 degrees 2θ. In some embodiments, Form G is characterized by an XRPD pattern comprising peaks at 9.1±0.5, 14.7±0.5, 16.0±0.5 and 17.2±0.5 degrees 2θ, and further comprising at least one additional peak at a position selected from the group consisting of 12.0±0.5, 13.5±0.5 and 17.7±0.5 degrees 2θ. In some embodiments, Form G is characterized by an XRPD pattern comprising peaks at 9.1±0.5, 12.0±0.5, 13.5±0.5, 14.7±0.5, 16.0±0.5, 17.2±0.5, and 17.7±0.5 degrees 2θ. In some embodiments, crystalline Form G is characterized by an XRPD pattern substantially as depicted in FIG. 5A.

In some embodiments, Form G is substantially free of other polymorphic forms. In some embodiments, Form G has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form G has a polymorphic purity of at least 90%. In some embodiments, Form G has a polymorphic purity of at least 92%. In some embodiments, Form G has a polymorphic purity of at least 94%. In some embodiments, Form G has a polymorphic purity of at least 96%. In some embodiments, Form G has a polymorphic purity of at least 98%. In some embodiments, Form G has a polymorphic purity of at least 99%.

### E. Crystalline Form D

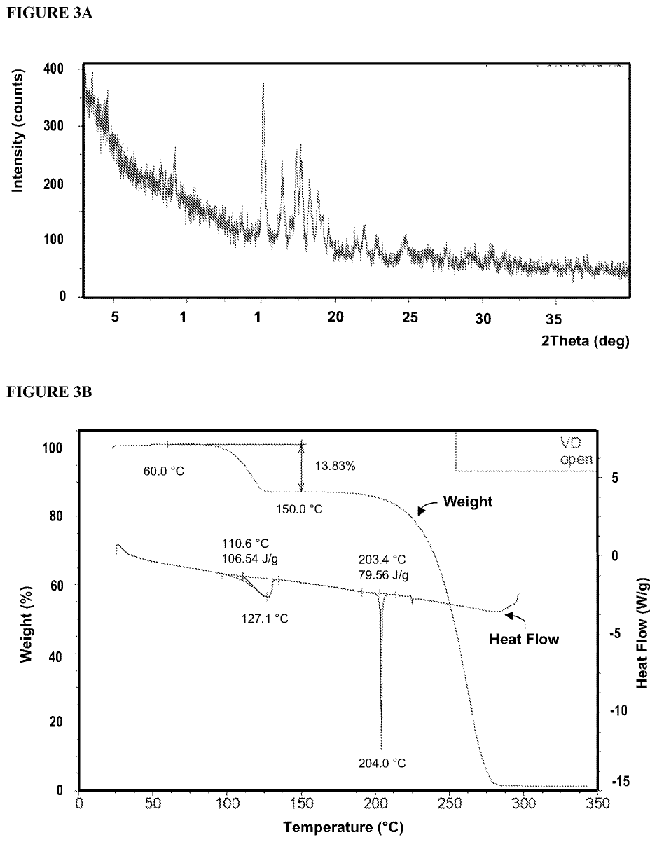

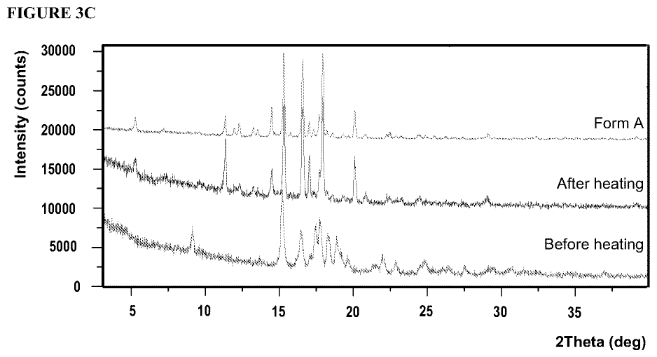

In some embodiments, the present disclosure relates to crystalline Form D of Compound 1. In some embodiments, the monohydrate crystalline form of Compound 1 is Form D. In some embodiments Form D has one or more characteristics selected from the group consisting of:
a) an XRPD pattern comprising peaks at 15.2±0.2, 17.4±0.2 and 17.7±0.2 degrees 2θ;
b) an XRPD pattern substantially as shown in FIG. 3A;
c) a DSC thermogram profile comprising an endothermic peak at about 204° C.;
d) a DSC thermogram profile comprising an endothermic peak at about 127° C.;
e) a DSC thermogram profile substantially as shown in FIG. 3B;
f) a TGA profile exhibiting a loss of at least about 14 wt % between about 60° C. and about 150° C.; and

g) a TGA profile substantially as shown in FIG. 3B.

In some embodiments, Form D is characterized by an XRPD pattern comprising peaks at 15.2±0.2, 17.4±0.2 and 17.7±0.2 degrees 2θ. In some embodiments, Form D is characterized by an XRPD pattern comprising peaks at 15.2±0.2, 17.4±0.2 and 17.7±0.2 degrees 2θ, and further comprising at least one additional peak at a position selected from the group consisting of 9.1±0.2, 16.4±0.2, 18.3±0.2, and 18.9±0.2 degrees 2θ. In some embodiments, Form D is characterized by an XRPD pattern comprising peaks at 15.2±0.2, 16.4±0.2, 17.4±0.2, 17.7±0.2, 18.3±0.2, and 18.9±0.2 degrees 2θ. In some embodiments, Form D is characterized by an XRPD pattern comprising peaks at 9.1±0.2, 15.2±0.2, 16.4±0.2, 17.4±0.2, 17.7±0.2, 18.3±0.2, and 18.9±0.2 degrees 2θ. In some embodiments, crystalline Form D is characterized by an XRPD pattern substantially as depicted in FIG. 3A.

In some embodiment, Form D is characterized by a thermo-gravimetric analysis (TGA) thermogram exhibiting that Form D loses at least about 14 wt % between about 60° C. and about 150° C. In some embodiment, Form D is characterized by a thermo-gravimetric analysis (TGA) thermogram exhibiting that Form D loses about 14-15 wt % between about 60° C. and about 150° C. In some embodiment, Form D is characterized by a thermo-gravimetric analysis (TGA) thermogram exhibiting that Form D loses about 14 wt % between about 60° C. and about 150° C. In some embodiments, Form D is characterized by a thermo-gravimetric analysis (TGA) thermogram substantially as depicted in FIG. 3B.

In some embodiments, Form D is characterized by a differential scanning calorimetry (DSC) thermogram substantially as depicted in FIG. 3B. In some embodiments, Form D is characterized by a DSC thermogram comprising an endothermic peak at about 204° C. In some embodiments, Form D is characterized by a DSC thermogram comprising an endothermic peak at about 127° C. In some embodiments, Form D is characterized by a DSC thermogram comprising an endothermic peak at about 127° C. and about 204° C. In some embodiments, Form D is characterized by a DSC thermogram comprising a desolvation endothermic peak at about 127° C. In some embodiments, Form D is characterized by a DSC thermogram comprising a melting endothermic peak at about 204° C.

In some embodiments, Form D is substantially free of other polymorphic forms. In some embodiments, Form D has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form D has a polymorphic purity of at least 90%. In some embodiments, Form D has a polymorphic purity of at least 92%. In some embodiments, Form D has a polymorphic purity of at least 94%. In some embodiments, Form D has a polymorphic purity of at least 96%. In some embodiments, Form D has a polymorphic purity of at least 98%. In some embodiments, Form D has a polymorphic purity of at least 99%.

### F. Crystalline Form E

In some embodiments, the crystalline form of Compound 1 is a solvate. In some embodiments, the solvate is a nBuOH solvate.

In some embodiments, the present disclosure relates to crystalline Form E of Compound 1. In some embodiments, the solvate crystalline form of Compound 1 is Form E. In some embodiments, the nBuOH solvate crystalline form of Compound 1 is Form E. In some embodiments Form E has one or more characteristics selected from the group consisting of:
a) an XRPD pattern comprising peaks at 4.9±0.2, 12.0±0.2, 12.2±0.2, and 17.0±0.2 degrees 2θ;
b) an XRPD pattern substantially as shown in FIG. 4A;
c) a DSC thermogram profile comprising an endothermic peak at about 203° C.;
d) a DSC thermogram profile comprising an endothermic peak at about 87° C.;
e) a DSC thermogram profile substantially as shown in FIG. 4B;
f) a TGA profile exhibiting a loss of at least about 7 wt % between about 22° C. and about 100° C.; and
f) a TGA profile substantially as shown in FIG. 4B.

In some embodiments, Form E is characterized by an XRPD pattern comprising peaks at 4.9±0.2, 12.0±0.2, 12.2±0.2, and 17.0±0.2 degrees 2θ. In some embodiments, Form E is characterized by an XRPD pattern comprising peaks at 4.9±0.2, 12.0±0.2, 12.2±0.2, and 17.0±0.2 degrees 2θ, and further comprising at least one additional peak at a position selected from the group consisting of 8.5±0.2, 14.9±0.2, 15.8±0.2, 18.2±0.2 and 21.4±0.2 degrees 2θ. In some embodiments, Form E is characterized by an XRPD pattern comprising peaks at 4.9±0.2, 12.0±0.2, 12.2±0.2, 17.0±0.2, and 21.4±0.2 degrees 2θ. In some embodiments, Form E is characterized by an XRPD pattern comprising peaks at 4.9±0.2, 8.5±0.2, 12.0±0.2, 12.2±0.2, 14.9±0.2, 15.8±0.2, 17.0±0.2, 18.2±0.2 and 21.4±0.2 degrees 2θ. In some embodiments, crystalline Form E is characterized by an XRPD pattern substantially as depicted in FIG. 4A.

In some embodiment, Form E is characterized by a thermo-gravimetric analysis (TGA) thermogram exhibiting that Form E loses at least about 7 wt % between about 22° C. and about 100° C. In some embodiments, Form E is characterized by a thermo-gravimetric analysis (TGA) thermogram substantially as depicted in FIG. 4B.

In some embodiments, Form E is characterized by a differential scanning calorimetry (DSC) thermogram substantially as depicted in FIG. 4B. In some embodiments, Form E is characterized by a DSC thermogram comprising an endothermic peak at about 203° C. In some embodiments, Form E is characterized by a DSC thermogram comprising an endothermic peak at about 87° C. In some embodiments, Form E is characterized by a DSC thermogram comprising an endothermic peak at about 87° C. and about 203° C. In some embodiments, Form E is characterized by a DSC thermogram comprising a desolvation endothermic peak at about 87° C. In some embodiments, Form E is characterized by a DSC thermogram comprising a melting endothermic peak at about 203° C.

In some embodiments, Form E is substantially free of other polymorphic forms. In some embodiments, Form E has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form E has a polymorphic purity of at least 90%. In some embodiments, Form E has a polymorphic purity of at least 92%. In some embodiments, Form E has a polymorphic purity of at least 94%. In some embodiments, Form E has a polymorphic purity of at least 96%. In some embodiments, Form E has a polymorphic purity of at least 98%. In some embodiments, Form E has a polymorphic purity of at least 99%.

### Pharmaceutical Compositions

In one aspect, the invention provides a pharmaceutical composition that is a solid dosage form comprising a solid form of Compound 1 as disclosed herein and a pharmaceutically acceptable excipient. In certain embodiments, the pharmaceutical composition comprises an effective amount of Compound 1. In certain embodiments, the pharmaceutical composition comprises a therapeutically effective amount of the Compound 1. In certain embodiments, the pharmaceutical composition comprises a prophylactically effective amount of Compound 1.

Solid dosage forms that are suitable for oral administration include solid formulations such as tablets, caplets, capsules, powders, lozenges (including liquid-filled), sachets and the like.

The solid dosage forms disclosed herein include a carrier, which may comprise one or more of pharmaceutically acceptable excipients, including, but not limited to, binders, surfactants, diluents or fillers, buffering agents, antiadherents, glidants, hydrophilic or hydrophobic polymers, retardants, stabilizing agents or stabilizers, disintegrants or superdisintegrants, dispersants, antioxidants, antifoaming agents, fillers, flavors, colorants, lubricants, sorbents, preservatives, plasticizers, coatings, or sweeteners, or mixtures thereof, as well as other excipients known in the art. For example, the excipient or excipients could be a binder such as microcrystalline cellulose, polyvinyl pyrrolidone, hydroxypropyl cellulose, low viscosity hydroxypropylmethylcellulose, gum tragacanth or gelatin; a diluent such as mannitol, microcrystalline cellulose, maltodextrin, starch or lactose, a disintegrating agent such as alginic acid, PRIMOGEL®, sodium starch glycolate, sodium croscarmellose, crospovidone, or corn starch; a lubricant such as magnesium stearate, sodium stearyl fumarate or glyceryl behenate; a glidant such as colloidal silicon dioxide; a preservative such as potassium sorbate or methyl paraben, a surfactant, such as sodium lauryl sulfate, sodium docusate, polysorbate 20, polysorbate 80, cetyl triethyl ammonium bromide, polyethylene oxide-polypropylene oxide copolymers, or CREMOPHOR® EL; an antioxidant such as butylhydroxytoluene, butyl hydroxyanisole, propyl gallate, ascorbic acid, tocopherol or tocopherol acetate, sodium sulphite, or sodium metabisulfite, a coating comprising one or more of hydroxypropylmethylcellulose, polyvinyl alcohol, methacrylate copolymers, cellulose acetate, hydroxypropylmethylcellulose acetate succinate, shellac and others, a sweetening agent such as sucrose, sucralose, acesulfame K, sodium aspartame or saccharin; or a flavoring agent such as peppermint, methyl salicylate, or orange flavoring. Any of the well-known pharmaceutical excipients may be incorporated in the dosage form and may be found in the FDA's Inactive Ingredients Guide, Remington: The Science and Practice of Pharmacy, Twenty-first Ed., (Pharmaceutical Press, 2005); Handbook of Pharmaceutical Excipients, Sixth Ed. (Pharmaceutical Press, 2009) all of which are incorporated by reference. In some embodiments, the solid dosage form disclosed herein include any one or more of the following excipients: microcrystalline cellulose low moisture, silicified microcrystalline cellulose low moisture type 90, mannitol, sodium starch glycolate, colloidal silicon dioxide, sodium stearyl fumarate, and opadry AMB II 88A180040 white. In some embodiments, the solid dosage form disclosed comprises microcrystalline cellulose low moisture, silicified microcrystalline cellulose low moisture type 90, mannitol, sodium starch glycolate, colloidal silicon dioxide, sodium stearyl fumarate, and opadry AMB II 88A180040 white. In some embodiments, the solid dosage form disclosed comprises microcrystalline cellulose low moisture, silicified microcrystalline cellulose low moisture type 90, mannitol, sodium starch glycolate, and sodium stearyl fumarate.

### Methods of Use

Compound 1 as described herein, is generally designed to be a positive allosteric modulator of NMDA function, and therefore is useful for the treatment and prevention of, e.g., CNS-related conditions in a subject.

In some embodiments, Compound 1 as described herein, is generally designed to penetrate the blood brain barrier (e.g., designed to be transported across the blood brain barrier). In certain embodiments of the present disclosure, Compound 1 acts as positive allosteric modulator (PAM) of NMDA and potentiates NMDA receptor function.

In one aspect, the disclosure provides a method for treating a disease, disorder or condition requiring positive allosteric NMDA modulation in a subject, comprising administering to the subject an effective amount of a crystalline form of Compound 1, or solid dosage form as disclosed herein.

In one aspect, the disclosure provides a method for treating a CNS-related condition in a subject, comprising administering to the subject an effective amount of a crystalline form of Compound 1, or a solid dosage form as disclosed herein.

In one aspect, the disclosure provides a method for preventing a disease, disorder or condition requiring positive allosteric NMDA modulation in a subject, comprising administering to the subject an effective amount of a crystalline form of Compound 1, or a solid dosage form as disclosed herein.

In one aspect, the disclosure provides a method for preventing a CNS-related condition in a subject, comprising administering to the subject an effective amount of a crystalline form of Compound 1, or a solid dosage form as disclosed herein.

In one aspect, the disclosure provides a method for inducing sedation or anesthesia in a subject, comprising administering to the subject an effective amount of a crystalline form of Compound 1, or a solid dosage as disclosed herein.

In one aspect, the disclosure provides a crystalline form of Compound 1, or a solid dosage as disclosed herein for use in treating a disease, disorder or condition requiring positive allosteric NMDA modulation in a subject.

In one aspect, the disclosure a crystalline form of Compound 1, or a solid dosage as disclosed herein for use in treating a CNS-related condition in a subject.

In one aspect, the disclosure provides a crystalline form of Compound 1, or a solid dosage as disclosed herein for use in preventing a disease, disorder or condition requiring positive NMDA modulation in a subject.

In one aspect, the disclosure provides a crystalline form of Compound 1, or a solid dosage as disclosed herein for use in preventing a CNS-related condition in a subject.

In one aspect, the disclosure provides a crystalline form of Compound 1, or a solid dosage as disclosed herein for use in inducing sedation or anesthesia in a subject.

In one aspect, the disclosure provides the use of a crystalline form of Compound 1, or a solid dosage as disclosed herein for the manufacture of a medicament for treating a disease, disorder or condition requiring positive allosteric NMDA modulation in a subject.

In one aspect, the disclosure provides the use of a crystalline form of Compound 1, or a solid dosage as disclosed herein for the manufacture of a medicament for treating a CNS-related condition in a subject.

In one aspect, the disclosure provides the use of a crystalline form of Compound 1, or a solid dosage as disclosed herein for the manufacture of a medicament for preventing a disease, disorder or condition requiring positive allosteric NMDA modulation in a subject.

In one aspect, the disclosure provides the use of a crystalline form of Compound 1, or a solid dosage as disclosed herein for the manufacture of a medicament for preventing a CNS-related condition in a subject.

In one aspect, the disclosure provides the use of a crystalline form of Compound 1, or a solid dosage as disclosed herein for the manufacture of a medicament for inducing sedation or anesthesia in a subject.

In some embodiments, the disorder is cancer. In some embodiments, the disorder is diabetes. In some embodiments, the disorder is a sterol synthesis disorder. In some embodiments, the disorder is a gastrointestinal (GI) disorder, e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis. In some embodiments, the disorder is inflammatory bowel disease.

In some embodiments, the disorder is Smith-Lemli-Opitz Syndrome (SLOS). In some embodiments, the disorder is desmosterolosis. In some embodiments, the disorder is sitosterolemia. In some embodiments, the disorder is cerebrotendinous xanthomatosis (CTX). In some embodiments, the disorder is Mevalonate Kinase Deficiency (MKD). In some embodiments, the disorder is SC4MOL gene mutation (SMO Deficiency). In some embodiments, the disorder is Niemann-Pick disease. In some embodiments, the disorder is autism spectrum disorder (ASD). In some embodiments, the disorder is associated with phenylketonuria

Exemplary conditions related to positive NMDA-modulation includes, but are not limited to, gastrointestinal (GI) disorder, e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis, and CNS conditions, e.g., as described herein.

Exemplary CNS conditions related to positive NMDA-modulation include, but are not limited to, adjustment disorders, anxiety disorders (including obsessive-compulsive disorder, posttraumatic stress disorder, social phobia, generalized anxiety disorder), cognitive disorders (including Alzheimer's disease and other forms of dementia (e.g., frontotemporal dementia)), dissociative disorders, eating disorders, mood disorders (including depression (e.g., postpartum depression), bipolar disorder, dysthymic disorder, suicidality), schizophrenia or other psychotic disorders (including schizoaffective disorder), sleep disorders (including insomnia), substance abuse-related disorders, personality disorders (including obsessive-compulsive personality disorder), autism spectrum disorders (including those involving mutations to the Shank group of proteins (e.g., Shank3)), neurodevelopmental disorders (including Rett syndrome), multiple sclerosis, sterol synthesis disorders, pain (including acute and chronic pain; headaches, e.g., migraine headaches), seizure disorders (including status epilepticus and monogenic forms of epilepsy such as Dravet's disease, and Tuberous Sclerosis Complex (TSC)), stroke, traumatic brain injury, movement disorders (including Huntington's disease and Parkinson's disease) and tinnitus.

In certain embodiments, a crystalline form of Compound 1, or a solid dosage as disclosed herein, can be used to induce sedation or anesthesia. In certain embodiments, the crystalline form of Compound 1, or a solid dosage as disclosed herein, is useful in the treatment or prevention of adjustment disorders, anxiety disorders, cognitive disorders, dissociative disorders, eating disorders, mood disorders, schizophrenia or other psychotic disorders, sleep disorders, substance-related disorders, personality disorders, autism spectrum disorders, neurodevelopmental disorders, sterol synthesis disorders, pain, seizure disorders, stroke, traumatic brain injury, movement disorders and vision impairment, hearing loss, and tinnitus. In some embodiments, the disorder is Huntington's disease. In some embodiments, the disorder is Parkinson's disease. In some embodiments, the disorder is an inflammatory disease (e.g., lupus).

In another aspect, provided is a method of treating or preventing brain excitability in a subject susceptible to or afflicted with a condition associated with brain excitability, comprising administering to the subject an effective amount of a crystalline form of Compound 1, or a solid dosage as disclosed herein.

In yet another aspect, the present disclosure provides a combination of a compound of the present disclosure, e.g., of a crystalline form of Compound 1, and another pharmacologically active agent. The compound provided herein can be administered as the sole active agent or they can be administered in combination with other agents. Administration in combination can proceed by any technique apparent to those of skill in the art including, for example, separate, sequential, concurrent and alternating administration.

### Diseases and Disorders

### Movement Disorders

Also described herein are methods for treating a movement disorder. As used herein, “movement disorders” refers to a variety of diseases and disorders that are associated with hyperkinetic movement disorders and related abnormalities in muscle control. Exemplary movement disorders include, but are not limited to, Parkinson's disease and Parkinsonism (defined particularly by bradykinesia), dystonia, chorea and Huntington's disease, ataxia, tremor (e.g., essential tremor), myoclonus and startle, tics and Tourette syndrome, Restless legs syndrome, stiff person syndrome, and gait disorders.

Tremor is an involuntary, at times rhythmic, muscle contraction and relaxation that can involve oscillations or twitching of one or more body parts (e.g., hands, arms, eyes, face, head, vocal folds, trunk, legs). Tremor includes hereditary, degenerative, and idiopathic disorders such as Wilson's disease, Parkinson's disease, and essential tremor, respectively; metabolic diseases (e.g., thyroid-parathyroid-, liver disease and hypoglycemia); peripheral neuropathies (associated with Charcot-Marie-Tooth, Roussy-Levy, diabetes mellitus, complex regional pain syndrome); toxins (nicotine, mercury, lead, CO, Manganese, arsenic, toluene); drug-induced (narcoleptics, tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor. Other forms of tremor include cerebellar tremor or intention tremor, dystonic tremor, essential tremor, orthostatic tremor, parkinsonian tremor, physiological tremor, psychogenic tremor, or rubral tremor.

Cerebellar tremor or intention tremor is a slow, broad tremor of the extremities that occurs after a purposeful movement. Cerebellar tremor is caused by lesions in or damage to the cerebellum resulting from, e.g., tumor, stroke, disease (e.g., multiple sclerosis, an inherited degenerative disorder).

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest.

Essential tremor or benign essential tremor is the most common type of tremor. Essential tremor may be mild and nonprogressive in some, and may be slowly progressive, starting on one side of the body but affect both sides within 3 years. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is often a precursor to Parkinson's disease and is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Physiological tremor can occur in normal individuals and have no clinical significance. It can be seen in all voluntary muscle groups. Physiological tremor can be caused by certain drugs, alcohol withdrawal, or medical conditions including an overactive thyroid and hypoglycemia. The tremor classically has a frequency of about 10 Hz.

Psychogenic tremor or hysterical tremor can occur at rest or during postural or kinetic movement. Patient with psychogenic tremor may have a conversion disorder or another psychiatric disease.

Rubral tremor is characterized by coarse slow tremor which can be present at rest, at posture, and with intention. The tremor is associated with conditions that affect the red nucleus in the midbrain, classical unusual strokes.

Parkinson's disease affects nerve cells in the brain that produce dopamine. Symptoms include muscle rigidity, tremors, and changes in speech and gait. Parkinsonism is characterized by tremor, bradykinesia, rigidity, and postural instability. Parkinsonism shares symptoms found in Parkinson's disease but is a symptom complex rather than a progressive neurodegenerative disease.

Dystonia is a movement disorder characterized by sustained or intermittent muscle contractions causing abnormal, often repetitive movements or postures. Dystonic movements can be patterned, twisting, and may be tremulous. Dystonia is often initiated or worsened by voluntary action and associated with overflow muscle activation.

Chorea is a neurological disorder characterized by jerky involuntary movements typically affecting the shoulders, hips, and face.

Huntington's Disease is an inherited disease that causes nerve cells in the brain to waste away. Symptoms include uncontrolled movements, clumsiness, and balance problems. Huntington's disease can hinder walk, talk, and swallowing.

Ataxia refers to the loss of full control of bodily movements, and may affect the fingers, hands, arms, legs, body, speech, and eye movements.

Myoclonus and Startle is a response to a sudden and unexpected stimulus, which can be acoustic, tactile, visual, or vestibular.

Tics are an involuntary movement usually onset suddenly, brief, repetitive, but non-rhythmical, typically imitating normal behavior and often occurring out of a background of normal activity. Tics can be classified as motor or vocal, motor tics associated with movements while vocal tics associated with sound. Tics can be characterized as simple or complex. For example, simple motor tics involve only a few muscles restricted to a specific body part.

Tourette Syndrome is an inherited neuropsychiatric disorder with onset in childhood, characterized by multiple motor tics and at least one vocal tic.

Restless Legs Syndrome is a neurologic sensorimotor disorder characterized by an overwhelming urge to move the legs when at rest.

Stiff Person Syndrome is a progressive movement disorder characterized by involuntary painful spasms and rigidity of muscles, usually involving the lower back and legs. Stiff-legged gait with exaggerated lumbar hyperlordosis typically results. Characteristic abnormality on EMG recordings with continuous motor unit activity of the paraspinal axial muscles is typically observed. Variants include “stiff-limb syndrome” producing focal stiffness typically affecting distal legs and feet.

Gait disorders refer to an abnormality in the manner or style of walking, which results from neuromuscular, arthritic, or other body changes. Gait is classified according to the system responsible for abnormal locomotion, and include hemiplegic gait, diplegic gait, neuropathic gait, myopathic gait, parkinsonian gait, choreiform gait, ataxic gait, and sensory gait.

### Mood Disorders

Also provided herein are methods for treating a mood disorder, for example clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior.

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Postnatal depression (PND), also known as postpartum depression (PPD), refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as) perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Suicidality, suicidal ideation, suicidal behavior refers to the tendency of an individual to commit suicide. Suicidal ideation concerns thoughts about or an unusual preoccupation with suicide. The range of suicidal ideation varies greatly from, e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

### Anxiety Disorders

Provided herein are methods for treating anxiety disorders. Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life-threatening illness (i.e., extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Epilepsy

Epilepsy is a brain disorder characterized by repeated seizures over time. Types of epilepsy can include, but are not limited to generalized epilepsy, e.g., childhood absence epilepsy, juvenile myoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Epileptogenesis

Epileptogenesis is a gradual process by which a normal brain develops epilepsy (a chronic condition in which seizures occur). Epileptogenesis results from neuronal damage precipitated by the initial insult (e.g., status epilepticus).

### Status Epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

### Seizure

A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well-known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures. In some embodiments, the seizure is a generalized seizure associated with Dravet Syndrome, Lennox-Gastaut Syndrome, Tuberous Sclerosis Complex, Rett Syndrome or PCDH19 Female Pediatric Epilepsy.

### Sterol Synthesis Disorders

In one embodiment, described herein are methods for treating a sterol synthesis disorder. Cholesterol has an essential rule in growth and development. It is a membrane lipid and a precursor to many molecules that play important roles in cellular growth and differentiation, protein glycosylation, and signaling pathways. Biosynthesis of cholesterol involves a number of enzymes and intermediates. Disorders resulting from a deficiency in any of the enzymes involved in cholesterol biosynthesis lead to the accumulation of intermediates and imbalance in biomolecules, resulting in disorders including congenital skeletal malformations, dysmorphic facial features, psychomotor retardation, and failure to thrive. In an embodiment, a sterol synthesis disorder or symptom of a sterol synthesis disorder can be treated by administering to a subject suffering from a sterol synthesis disorder a compound described herein, such as a NMDA receptor modulating compound as described herein. Additional disorders are described below.

### Smith-Lemli-Opitz Syndrome

In one embodiment, described herein are methods for treating Smith-Lemli-Opitz Syndrome (or SLOS, or 7-dehydrocholesterol reductase deficiency). SLOS is an inborn error of cholesterol synthesis. In addition to microcephaly, moderate to severe intellectual disability, sensory hypersensitivity, stereotyped behaviors, dysmorphic facial features, and syndactyly of the second/third toes, a feature of the disease is reduced cerebrosterol (24(S)-hydroxycholesterol) levels. SLOS is an autosomal recessive genetic condition resulting from deficiency in the final enzyme of the cholesterol synthesis pathway and causes low or low-normal plasma cholesterol levels and increased 7- and 8-dehydrocholesterol (DHC; 7DHC and 8DHC) levels. Common therapies currently used include dietary cholesterol supplementation, treatment with 3-hydroxy-3-methylglutaryl coenzyme A reductase inhibitors (HMG CoA reductase inhibitors, also known as statins), and treatment with agents that enhance cholesterol production and/or accretion; and to decrease the accumulation of 7DHC and 8DHC, the potentially toxic precursors of cholesterol.

### Desmosterolosis

Desmosterolosis is a deficiency in desmosterol reductase and has a similar phenotype to SLOS. In one embodiment, described herein are methods for treating desmosterolosis with the compound described herein.

### Sitosterolemia

Sitosterolemia is a rare autosomal recessive disorder caused by mutations in two ATP-binding cassette (ABC) transporter genes (ABCG5 and ABCG8). Sitosterolemia enhances the absorption of plant sterols and cholesterol from the intestines. Patients typically present with tendon and tuberous xanthomas and premature coronary artery disease. In one embodiment, described herein are methods for treating sitosterolemia with the compound described herein.

### Cerebrotendinous Xanthomatosis (CTX)

In one embodiment, described herein are methods for treating cerebrotendinous xanthomatosis (also referred to as cerebral cholesterosis, or Van Bogaert-Scherer-Epstein syndrome) with the compound described herein. CTX can be caused by a mutation in the CYP27A1 gene, which produces the sterol 27-hydroxylase enzyme. Sterol 27-hydroxylase metabolizes cholesterol into bile acids (e.g., chenodeoxycholic acid) that are important in the absorption of fats in the intestine. Enzyme dysfunction can lead to cholesterol accumulation in tissues. CTX is characterized by childhood diarrhea, cataracts, tendon xanthomas, reduced mental capability and abnormal movements in adults.

### Mevalonate Kinase Deficiency Syndromes (MKD)

Mevalonate Kinase Deficiency (also referred to as mevalonic aciduria (a more severe form of MKD), or Hyper IgD Syndrome (HIDS, or hyperimmunoglobulinemia D) with period fever syndrome (a more benign form of MKD)) causes an accumulation of mevalonic acid in the urine as a result of insufficient activity of mevalonate kinase. MKD can result in developmental delay, hypotonia, anemia, hepatosplenomegaly, dysmorphic features, mental retardation, and overall failure to thrive. Mevalonic aciduria is characterized by delayed physical and mental development, failure to thrive, recurrent episodes of fever with vomiting and diarrhea, enlarged liver, spleen and lymph nodes, microcephaly (small head size), cataract, low muscle tone, short statute, distinct facial features, ataxia, and anemia. HIDS is characterized by recurrent episodes of fever associated with swollen lymph nodes, joint pain, gastrointestinal issues and skin rash. In one embodiment, described herein are methods for treating MKD with the compound described herein.

### SC4MOL Gene Mutation (SMO Deficiency)

SC4MOL gene deficiency is a genetic disorder in the cholesterol biosynthesis pathway (e.g., mutations in the SC4MOL gene encoding a novel sterol oxidase). SC4MOL deficiency is characterized by the accumulation of dimethyl and monomethyl sterols that can be detected in blood, skin flakes or primary skin fibroblasts. In one embodiment, described herein are methods for treating SMO deficiency with the compound described herein.

### Niemann-Pick Disease

Niemann-Pick disease is a lysosomal storage disease resulting from a genetic mutation that affects metabolism. Niemann-Pick disease leads to abnormal accumulation of cholesterol and other fatty substances (lipids) due to an inability of the body to transport the substances. The accumulation damages the affected areas.

### Autism

In one embodiment, described herein are methods for treating autism spectrum disorder or autism. Autism spectrum disorder (ASD) and autism refer to a group of complex disorders of brain development. Autism is typically characterized by difficulties in social interaction, for example in verbal and nonverbal communication. Repetitive behaviors are also often seen in individuals having autism. Autism can be associated with intellectual disability, difficulties in motor coordination and attention and physical health issues, e.g., sleep and gastrointestinal disturbances. Individuals having autism can also excel in visual skills, music, math and art. Autism can refer to autistic disorder, childhood disintegrative disorder, pervasive developmental disorder-not otherwise specified (PDD-NOS), and Asperger syndrome. Autism also refers to monogenetic causes of autism such as synaptopathies, e.g., Rett syndrome, Fragile X syndrome, Angelman syndrome.

Disorders Associated with Phenylketonuria

In one embodiment, described herein are methods for treating disorders associated with phenylketonuria (e.g., cognitive disorders) with the compound described herein. Phenylketonuria can lead to hypocholesterolemia and lowered vitamin D status. Total and low-density cholesterols and 25-hydroxy vitamin D have been found to be decreased in subjects suffering from phenylketonuria as compared with subjects not suffering from phenylketonuria (Clin. Chim. Acta 2013, 416:54-59). 24S-hydroxycholesterol and 27S-hydroxycholesterol and 7α-hydroxycholesterol (e.g., representing peripheral and hepatic cholesterol elimination, respectively) have been shown to be significantly decreased in subjects suffering from phenylketonuria, while 7β-hydroxycholesterol (e.g., reflecting oxidative stress) was increased significantly in subjects suffering from phenylketonuria. Changes in the levels of 24S-OHC and 7β-hydroxycholesterol correlate with phenylalanine level, and 27S-hydroxycholesterol levels may correlate with the 25-hydroxy vitamin D level in subjects suffering from phenylketonuria.

### Method of Making Crystalline Forms

In one aspect, the disclosure provides a method of making a crystalline form of Compound 1 as disclosed herein.

In some embodiments, the disclosure provides a method of making a crystalline form of Compound 1 comprising the steps of: a) dissolving Compound 1 in at least one solvent to form a solution; b) adding an anti-solvent until a precipitate forms; and c) isolating the precipitate. In some embodiments, the solvent is selected from the group consisting of THF, 1,4-dioxane and MEK. In some embodiments, the anti-solvent is selected from the group consisting of H₂O and ACN.

In some embodiments, the disclosure provides a method of making a crystalline form of Compound 1 comprising the steps of: a) adding Compound 1 to a solvent to form a mixture; b) aging the mixture at room temperature to precipitate solids; and c) isolating the solids. In some embodiments, the solvent is selected from the group consisting of DME, Acetone, nBuOH, EtOAc, IPAc, BuOAc, diethyl ether, and ACN.

In some embodiments, the disclosure provides a method of making a crystalline form of Compound 1 comprising the steps of: a) adding Compound 1 to a solvent to form a mixture; b) aging the mixture at about 40° C. to precipitate solids; and c) isolating the solids. In some embodiments, the solvent is selected from the group consisting of IPAc, BuOAc, ACN, and n-heptane.

In some embodiments, the disclosure provides a method of making a crystalline form of Compound 1 comprising the steps of: a) adding Compound 1 to a volatile solvent to form a mixture; b) aging the mixture at room temperature to allow the volatile solvent vapor to interact with Compound 1 and to precipitate solids; and c) isolating the solids. In some embodiments, the solvent is selected from the group consisting of DME, MEK, diethyl ether and t-BuOH.

In some embodiments, the disclosure provides a method of making a crystalline form of Compound 1 comprising the steps of: a) adding Compound 1 to a solvent to form a solution; b) adding the solution to an anti-solvent; c) aging the mixture at room temperature to precipitate solids; and c) isolating the solids. In some embodiments, the solvent is BuOH. In some embodiments, the anti-solvent is n-Heptane.

In some embodiments, the disclosure provides a method of making a crystalline form of Compound 1 comprising the steps of: a) adding Compound 1 to solvent to form a mixture; b) filtering the mixture to provide a filtrate; c) aging the filtrate at room temperature; d) allowing solids to precipitate; and e) isolating the solids. In some embodiments, the solvent is selected from the group consisting of IPAc, and t-BuOH.

In some embodiments, the disclosure provides a method of making a crystalline form of Compound 1 comprising the steps of: a) adding Compound 1 to solvent to form a mixture; b) heating the mixture to about 50° C.; c) filtering the mixture to provide a filtrate; c) cooling the filtrate to about −20° C.; d) allowing solids to precipitate; and e) isolating the solids. In some embodiments, the solvent is acetone.

In some embodiments, the disclosure provides a method of making a crystalline form of Compound 1 comprising the steps of: a) heating Compound 1 to about 205° C.; b) cooling the heated Compound 1 to about 25° C. to provide solids; and c) isolating the solids. In some embodiments, the cooling in step b) is at a rate selected from the group consisting of 2° C./min, 10° C./min and 50° C./min.

In some embodiments, the disclosure provides a method of making a crystalline form of Compound 1 comprising the steps of: a) adding Compound 1 to solvent to form a mixture; b) aging the mixture at about 90° C. to precipitate solids; and d) isolating the solids. In some embodiments, the solvent is n-heptane.

In some embodiments, the disclosure provides a method of making a crystalline form of Compound 1 comprising the steps of: a) adding Compound 1 to solvent to form a mixture; b) grinding the mixture to provide solids; and d) isolating the solids. In some embodiments, the solvent is acetone/MIBK.

In some embodiments, the disclosure provides a method of making a crystalline form of Compound 1 comprising the steps of: a) adding Form E of Compound 1 to a first solvent to form a mixture; b) concentrating the mixture by distillation at atmospheric pressure; c) cooling the mixture to about 80-85° C.; d) adding a second solvent to the mixture and maintaining the temperature of the mixture at about 80-85° C. for at least 1 hour; e) concentrating the mixture by distillation at atmospheric pressure; f) cooling the mixture to about 20° C. to precipitate solids; and g) isolating the solids. In some embodiments, the first solvent is heptane. In some embodiments, the second solvent is n-BuOH.

In some embodiments, the disclosure provides a method of making a crystalline form of Compound 1 comprising the steps of: a) adding Form E of Compound 1 to heptane to form a mixture; b) concentrating the mixture by distillation at atmospheric pressure; c) cooling the mixture to about 80-85° C.; d) adding n-BuOH to the mixture and maintaining the temperature of the mixture at about 80-85° C. for at least 1 hour; e) concentrating the mixture by distillation at atmospheric pressure; f) cooling the mixture to about 20° C. to precipitate solids; and g) isolating the solids.

In some embodiments, the disclosure provides a method of making a crystalline form of Compound 1 comprising the steps of: a) adding Form E of Compound 1 to a solvent to form a mixture; b) heating the mixture to about 50° C.; c) adding antisolvent; d) adding Form X of Compound 1 seeds and aging the mixture at about 50° C.; e) cooling the mixture to about 20° C. over at least 3 hours and holding the mixture at 20° C. (wet milling optional) to precipitate solids; and f) isolating the solids. In some embodiments, the solvent is acetone. In some embodiments, the anti-solvent is acetone/water. In some embodiments, the anti-solvent is acetone/water (1:1 v/v).

In some embodiments, the disclosure provides a method of making a crystalline form of Compound 1 comprising the steps of: a) adding Form E of Compound 1 to acetone to form a mixture; b) heating the mixture to about 50° C.; c) adding acetone/water antisolvent (1:1 v/v); d) adding Form X of Compound 1 seeds and aging the mixture at about 50° C.; e) adding acetone/water antisolvent; f) cooling the mixture to about 20° C. over at least 3 hours and holding the mixture at 20° C. (wet milling optional) to precipitate solids; and g) isolating the solids.

In some embodiments, the disclosure provides a method of making a crystalline form of Compound 1 comprising the steps of: a) adding crude Compound 1 to a solvent to form a mixture and heating to about 80° C.; b) concentrating the mixture via distillation; c) adding solvent to the mixture; d) adjusting the temperature of the mixture to about 80° C.; e) adding Form E of Compound 1 seeds; f) cooling the mixture to about 15-25° C.; g) aging the mixture at about 15-25° C. for about 4-18 hours to precipitate solids; and h) isolating the solids. In some embodiments, the solvent is n-BuOH.

In some embodiments, the disclosure provides a method of making a crystalline form of Compound 1 comprising the steps of: a) adding crude Compound 1 to n-BuOH to form a mixture and heating to about 80° C.; b) concentrating the mixture via distillation; c) adding n-BuOH to the mixture; d) adjusting the temperature of the mixture to about 80° C.; e) adding Form E of Compound 1 seeds; f) cooling the mixture to about 15-25° C.; g) aging the mixture at about 15-25° C. for about 4-18 hours to precipitate solids; and h) isolating the solids.

### EXAMPLES

In order that the invention described herein may be more fully understood, the following examples are set forth. The Examples are offered to illustrate the crystalline solid forms provided herein and are not to be construed in any way as limiting their scope.

### Example 1

### Instruments and Methodology

### A. X-Ray Powder Diffraction (XRPD)

A PANalytical Empyrean X-ray powder diffractometer was used to collect X-ray diffraction patters as described herein. The diffraction peak position was calibrated using a PANalytical Si reference standard disc. The XRPD parameters used are listed in Table 1.

TABLE 1
Parameters for XRPD test

| Parameters | Reflection Mode |
|---|---|
| X-Ray wavelength | Cu, kα |
| | Kα1 (Å): 1.540598, Kα2 (Å): 1.544426 |
| | Kα2/Kα1 intensity ratio: 0.50 |
| X-Ray tube setting | 45 kV, 40 mA |
| Divergence slit | Fixed ⅛° |
| Scan mode | Continuous |
| Scan range | 3-40 |
| (° 2θ) | |
| Scan step time [s] | 18.87 |
| Step size | 0.0131 |
| (° 2θ) | |
| Test Time | 4 min 15 s |

### B. Thermo-Gravimetric Analysis (TGA) and Differential Scanning Calorimetry (DSC)

TGA data was collected using a TA Discovery 550/Q500 TGA from TA Instrument. DSC was performed using a TA Q2000 DSC from TA Instrument. DSC was calibrated with Indium reference standard and the TGA was calibrated using nickel reference standard. Detailed parameters are provided in Table 2:

TABLE 2
Parameters for TGA and DSC test

| Parameters | TGA | DSC |
|---|---|---|
| Method | Ramp | Ramp |
| Sample pan | Platinum open | Aluminum, crimped |

| | |
|---|---|
| Temperature | RT - desired temperature |
| Heating rate | 10° C./min |
| Purge gas | N₂ |

### C. Polarized Light Microscopy (PLM) and Hot Stage

Polarized light microscopic picture was captured on Nikon DS-Fi2 upright microscope at room temperature. Hot stage was performed on Linkam.

### D. Chemical Purity Determination (HPLC)

Purity analysis and stoichiometry were performed using an Agilent 1100 HPLC as detailed in Table 3:

TABLE 3
HPLC method for purity test

| HPLC | Agilent 1100 with DAD detector | |
|---|---|---|
| Column | Agilent Zorbax Eclipse Plus C18, | |
| | 150 × 4.6 mm, 3.5 μm | |
| Mobile phase | A: H₂O | |
| | B: Acetonitrile | |
| | Time (min) | % B |
| Gradient table | 0.0 | 50 |
| | 15.0 | 100 |
| | 25.0 | 100 |
| | 26.0 | 50 |
| | 30.0 | 50 |

| | |
|---|---|
| Run time | 30.0 min |
| Post time | 0.0 min |
| Flow rate | 1.2 mL/min |
| Injection volume | 5 μL |
| Detector wavelength | UV at 205 nm |
| Column temperature | 30° C. |
| Sampler | RT |
| temperature | |
| Diluent | MeOH |

### E. Dynamic Vapor Sorption (DVS)

DVS was measured using a SMS (Surface Measurement Systems) DVS Intrinsic. The relative humidity at 25° C. was calibrated against deliquescence point of LiCl, Mg(NO₃)₂ and KCl. The parameters used in the DVS test are provided in Table 4.

TABLE 4
Parameters for DVS test

| Item | Value | |
|---|---|---|
| Temperature | 25° | C. |
| Sample size | 10~20 | mg |
| Gas and flow rate | N₂, 200 mL/min | |
| dm/dt | 0.002%/min | |
| Min. dm/dt stability duration | 10 | min |
| Max. equilibrium time | 180 | min |

| | |
|---|---|
| RH range | 0% RH to 95% RH to 0% RH |
| RH step size | 10% RH from 0% RH to 90% RH |
| | 5% RH from 90% RH to 95% RH |
| | 5% RH from 95% RH to 90% RH |
| | 10% RH from 90% RH to 0% RH |

### F. Nuclear Magnetic Resonance (NMR)

Solution NMR spectra were collected on a Bruker 400M NMR Spectrometer. Samples were prepared in DMSO-d₆.

### Example 2

### Abbreviations

The solvent abbreviations are provided in Table 5.

TABLE 5
Abbreviations

| Abbreviation | Solvent | Abbreviation | Solvent |
|---|---|---|---|
| MeOH | Methanol | THF | Tetrahydrofuran |
| EtOH | Ethanol | 2-MeTHF | 2- |
| | | | Methyltetrahydrofuran |
| DME | Dimethoxyethane | n-PrOH | Isopropyl alcohol |
| DMF | Dimethylformamide | NMP | N-Methyl-2-pyrrolidone |
| n-BuOH | n-Butanol | MIBK | Methyl isobutyl ketone |
| CPME | Cyclopentyl methyl | MEK | Methyl Ethyl Ketone |
| | ether | | |
| BuOAc | Butyl acetate | DCM | Dichloromethane |
| DCM | Dichloromethane | t-BuOH | t-Butanol |
| IPA | Isopropyl alcohol | DMSO | Dimethyl sulfoxide |
| EtOAc | Ethyl acetate | CHCl₃ | Trichloromethane |
| IPAc | Isopropyl acetate | ACN | Acetonitrile |
| MTBE | Methyl tert-butyl ether | | |

### Example 3

### Polymorph Screening

Polymorph screening experiments were performed using different crystallization or solid transition methods. The methods utilized and crystalline forms identified are summarized in Table 6.

TABLE 6
Methods and Crystalline Forms

| Method | Crystalline Form |
|---|---|
| Anti-solvent addition | A |
| Slurry at RT/40° C./5° C. | A, E |
| Solid vapor diffusion | A |
| Liquid vapor diffusion | E |
| Slow cooling | A |
| Slow evaporation | B |
| Heating-cooling | A |
| High temperature slurry | A |
| Wet grinding | A |

### A. Anti-Solvent Addition

About 15 mg of starting material (Compound 1) was dissolved in 0.1-0.9 mL solvent to obtain a clear solution. The solution was magnetically stirred followed by addition of 0.2 mL anti-solvent per step until a precipitate appeared or the total amount of anti-solvent reached 15.0 mL. Results in Table 7 (below) showed the obtained forms.

TABLE 7
Summary of anti-solvent addition experiments

| Solvent | Anti-solvent | Solid Form |
|---|---|---|
| THF | ACN | A* |
| 1,4-Dioxane | ACN | A* |
| MEK | ACN | A* |

*solids were obtained after stirring the clear solution at −20° C.

### B. Slurry at Room Temperature

Slurry conversion experiments were conducted at room temperature in different solvent systems. About 15 mg of starting material (Compound 1) was suspended in 0.2 mL of solvent in a glass vial. After the suspension was stirred magnetically for 7 days at room temperature, the remaining solids were isolated. Results are summarized in Table 8 below.

TABLE 8
Summary of slurry conversion experiments at room temperature

| Solvent (v:v) | Solid Form |
|---|---|
| DME | A |
| Acetone | A |
| n-BuOH | E |
| EtOAc | A |
| IPAc | A |
| BuOAc | A |
| Diethyl ether | A |
| ACN | A |

### C. Slurry at 40° C.

Slurry conversion experiments were also conducted at 40° C. in different solvent systems. About 10 mg of starting material (Compound 1) was suspended in 0.3 mL of solvent in a glass vial. After the suspension was stirred for about 7 days at 40° C., the remaining solids were isolated. Results are summarized in Table 9.

TABLE 9
Summary of slurry conversion experiments at 40° C.

| Solvent | Solid Form |
|---|---|
| IPAc | A |
| BuOAc | A |
| ACN | A |
| n-Heptane | A |

### D. Solid Vapor Diffusion

Solid vapor diffusion experiments were conducted using different solvents. Approximate 10 mg of starting material (Compound 1) was weighed into a 3-mL vial, which was placed into a 20-mL vial with 2 mL of volatile solvent. The 20-mL vial was sealed with a cap and kept at RT for 7 days allowing solvent vapor to interact with sample. The results are summarized in Table 10.

TABLE 10
Summary of solid vapor diffusion experiments

| Solvent | Solid Form |
|---|---|
| DME | A |
| MEK | A |
| Diethyl ether | A |
| t-BuOH | A |

### E. Liquid Vapor Diffusion

Approximate 15 mg of starting material (Compound 1) was dissolved in appropriate solvent to obtain a clear solution in a 3-mL vial. This solution was then placed into a 20-mL vial with 3 mL of volatile solvents. The 20-mL vial was sealed with a cap and kept at room temperature allowing sufficient time for organic vapor to interact with the solution. The results are summarized in Table 11.

TABLE 11
Summary of liquid vapor diffusion experiments

| Solvent | Anti-solvent | Solid Form |
|---|---|---|
| BuOH | n-Heptane | E |

### F. Slow Evaporation

Briefly, 15 mg of starting material (Compound 1) was dissolved in 0.5~1.5 mL of solvent in a 3-mL glass vial. If no dissolution was achieved, suspensions were filtered using a PTFE membrane (pore size of 0.20 μm) and the filtrates were used for the following steps. The visually clear solutions were covered by Parafilm® with 6 pinholes and subjected to evaporation at room temperature. The results are summarized in Table 12.

TABLE 12
Summary of slow evaporation experiments

| Solvent | Solid Form |
|---|---|
| t-BuOH | B |

### G. Slow Cooling

About 20 mg of starting material (Compound 1) was suspended in 0.5~1.0 mL of solvent in a 3-mL glass vial at room temperature. The suspension was then heated to 50° C., equilibrated for about two hours and filtered using a PTFE membrane (pore size of 0.22 μm). Filtrates were slowly cooled down to 5° C. at a rate of 0.1° C./min. No solid was obtained for any system and the solutions were then transferred to −20° C. If no precipitation observed, the solutions were subjected to evaporation at RT. The results are summarized in Table 13.

TABLE 13
Summary of slow cooling experiments

| Solvent (v:v) | Solid Form |
|---|---|
| Acetone | A |

### H. Heating Experiment

About 10 mg of starting material (Compound 1) was weighed into a DSC pan. The sample was heated to 205° C. at 30° C./min and cooled to 25° C. at different rate (2, 10, 50° C./min). The heated sample was tested by XRPD. The results are summarized in Table 14.

TABLE 14
Summary of heating-cooling experiments

| Cooling rate (° C./min) | Solid Form |
|---|---|
| 2 | A |
| 10 | A |
| 50 | A |

### I. High Temperature Slurry

High temperature slurry conversion experiments were conducted at 90° C. in different solvent systems. About 100 mg of starting material (Compound 1) was suspended in 0.3 mL of solvent in a glass vial. After the suspension was stirred magnetically for 24 hrs at 90° C., the remaining solids were isolated for XRPD analysis. The results are summarized in Table 15.

TABLE 15
Summary of high temperature slurry

| Solvent | Solid Form |
|---|---|
| n-Heptane | A |

### J. Wet Grinding

About 20 mg of Form A and 0.2 mL solvent were added into the mortal and grind for 10 min. The remaining solids were tested by XRPD. The results are summarized in Table 16.

TABLE 16
Summary of wet grinding

| Solvent (1:1, v/v) | Solid Form |
|---|---|
| Acetone/MIBK | A |

### Example 4

Several crystal forms were obtained via the screening, including two anhydrates (Forms A and B), two hydrates (Form X and D), a solvate (Form E). The detailed characterization data of the various forms is provided in Table 17.

TABLE 17
Characterization of crystal forms

| Crystal | | | Endo. in | | |
|---|---|---|---|---|---|
| Form | | Wt. | DSC | HPLC | |
| (Sample | Preparation | Loss | (peak, | Purity | Speculated |
| ID) | Conditions | (%) | ° C.) | (Area %) | Form |
| Form A | Starting | 0.04 | 203.0 | 98.43 | Anhydrate |
| | material | (190° C.) | | | |
| Form B | Heating | 0.2 | 203.7 | 98.50 | Anhydrate |
| | Form X | (150° C.) | | | |
| Form D | Vacuum | 13.8 | 127.1, | 98.47 | Hydrate |
| | dry of | (150° C.) | 204.0 | | |
| | Form X | | | | |
| Form E | Liquid | 7.2 | 86.7, | 98.45 | BuOH |
| | vapor | (100° C.) | 203.1 | | solvate |
| | diffusion | | | | |
| | in BuOH/ | | | | |
| | n-heptane | | | | |

The XRPD patterns for each form is provided in FIGS. 1A-5A. These crystal forms exhibited the appropriate properties (e.g., chemical stability, thermal stability, solubility, hygroscopicity, particle size, yield, impurity content during crystallization, drying properties, milling properties, and stability during tableting) for preparing solid dosage forms.

### Example 5

### Preparation and Characterization of Form A

Form A was used directly for the polymorph screening. The XRPD pattern for Form A is provided in FIG. 1A and characteristic peaks are summarized in Table 18. Form A had a chemical purity of about 98% as detected by HPLC. TGA and DSC data for Form A is provided in FIG. 1B and showed a 0.04% weight loss up to 190° C., and an endotherm at 203.0° C. (peak temperature) due to melting. In view of these results, Form A was deemed to be an anhydrate.

The crystals disappeared after heating to 205° C., suggestive of evaporation post melting. See FIG. 1C. To investigate the solid form stability as a function of humidity, a DVS isotherm plot of Form A was collected at 25° C. The results showed a water uptake of 0.05% at 25° C./80% RH, with no form change after DVS test. See FIGS. 1D and 1E, suggesting Form A was non-hygroscopic.

TABLE 18
Observed XRPD peaks for Form A

| Pos. [°2 Theta] | d-spacing [Å] | Rel. Int. [%] |
|---|---|---|
| 5.3 | 16.78 | 16.8 |
| 11.3 | 7.81 | 24.4 |
| 12.0 | 7.40 | 8.6 |
| 12.3 | 7.20 | 15.2 |
| 13.2 | 6.69 | 10.9 |
| 13.6 | 6.53 | 8.1 |
| 14.5 | 6.12 | 31.4 |
| 15.3 | 5.80 | 100.00 |
| 16.6 | 5.35 | 96.1 |
| 17.0 | 5.22 | 18.0 |
| 17.7 | 5.01 | 26.9 |
| 17.9 | 4.96 | 92.0 |
| 20.1 | 4.42 | 34.3 |

### Example 6

### Preparation and Characterization of Form B

Form B was generated via heating of Form X. The XRPD pattern for Form B is shown in FIG. 2A and characteristic peaks are summarized in Table 19. Form B had a chemical purity of about 99% as detected by HPLC. TGA and DSC data for Form B is provided in FIG. 2B and showed a weight loss of 0.2% up to 150° C. The DSC data showed a sharp melting peak at 203.7° C. (peak temperature). In view of these results, Form B was deemed to be an anhydrate.

TABLE 19
Observed XRPD peaks for Form B

| Pos. [°2Theta] | d-spacing [Å] | Rel. Int. [%] |
|---|---|---|
| 7.7 | 11.45 | 10.1 |
| 10.9 | 8.09 | 32.9 |
| 14.4 | 6.16 | 57.1 |
| 15.4 | 5.75 | 32.6 |
| 16.8 | 5.28 | 100.0 |
| 18.0 | 4.93 | 17.9 |
| 18.5 | 4.80 | 56.4 |
| 19.0 | 4.67 | 45.3 |
| 23.1 | 3.86 | 12.0 |

### Example 7

### Preparation Form X

Form X was generated via slurry of Form A in EtOH/H₂O (0.927/0.073, a_{w}=0.4) at RT.

### Example 8

### Preparation and Characterization of Form D

Form D was generated via vacuum dry of Form X. The XRPD pattern for Form D is provided in FIG. 3A and characteristic peaks are summarized in Table 20. Form D had a chemical purity of about 98% as detected by HPLC. TGA and DSC data for Form D is provided in FIG. 3B and showed a weight loss of 13.8% up to 150° C. and a desolvation endotherms at 127.1° C. (peak temperature) before a possible melting peak at 204.0° C. (peak temperature). After heating to 150° C. and then cooling down to room temperature under nitrogen protection and re-exposure to ambient conditions, Form D converted to Form A (see FIG. 3C).

TABLE 20
Observed XRPD peaks for Form D

| Pos. [°2Theta] | d-spacing [Å] | Rel. Int. [%] |
|---|---|---|
| 9.1 | 9.69 | 30.5 |
| 15.2 | 5.84 | 100.0 |
| 16.4 | 5.40 | 45.9 |
| 17.4 | 5.10 | 51.6 |
| 17.7 | 5.00 | 63.2 |
| 18.3 | 4.86 | 46.4 |
| 18.9 | 4.70 | 41.9 |
| 19.6 | 4.52 | 14.9 |
| 22.0 | 4.04 | 20.9 |
| 22.9 | 3.89 | 12.8 |
| 24.8 | 3.58 | 15.7 |

### Example 9

### Preparation and Characterization of Form E

Form E was generated via liquid diffusion in BuOH/n-heptane of Form A at RT. The XRPD pattern for Form E is provided in FIG. 4A and characteristic peaks are summarized in Table 21. Form E had a chemical purity of about 98% as detected by HPLC. TGA and DSC data for Form E is provided in FIG. 4B and showed a weight loss of 7.2% up to 100° C. and a desolvation endotherm at 86.7° C. (peak temperature) before possible melting peak at 203.1° C. (peak temperature). After heating to 140° C. and then cooling down to room temperature under nitrogen protection and re-exposure to ambient conditions, Form E converted to Form B (see FIG. 4C). As evidenced by the presence of n-butanol methylene proton at ~3.7 ppm (1.5:1, molar ratio of n-butanol/API) detected by ¹H NMR in FIG. 4D, Form E was deemed to be an n-butanol solvate.

TABLE 21
Observed XRPD peaks for Form E

| Pos. [°2Theta] | d-spacing [Å] | Rel. Int. [%] |
|---|---|---|
| 4.9 | 17.94 | 75.1 |
| 6.6 | 13.46 | 13.4 |
| 8.5 | 10.43 | 22.7 |
| 12.0 | 7.36 | 67.7 |
| 12.2 | 7.26 | 90.4 |
| 14.2 | 6.25 | 13.8 |
| 14.9 | 5.95 | 28.7 |
| 15.8 | 5.61 | 25.90 |
| 17.0 | 5.22 | 100.0 |
| 18.2 | 4.88 | 29.1 |
| 21.4 | 4.16 | 33.3 |
| 22.8 | 3.90 | 17.4 |
| 24.2 | 3.67 | 12.4 |

### Example 10

### Preparation and Characterization of Form F

### Form F was Generated Via the Following Process:

1.1 Conversion at 80° C. with Different Concentration

Three slurries were setup at 50, 100, and 150 g of BuOH solvate of Compound 1/L of heptane, in 1-2 mL scale. The slurries were placed on a temperature controlled shaker at 80° C. and agitated overnight at 600 rpm. The wet-cakes in the slurries post overnight conditioning were tested by XRPD, which all gave patterns conforming to the target anhydrous Form F.

The slurry of 150 g/L was very thick like a cake, while the slurry at 100 g/L and 50 g/L were homogeneous and free-flowing slurries. 100 g/L is a suitable concentration for the slurry conversion process.

### 1.2 Conversion Kinetics at 80° C.

The conversion kinetics at 80° C. was probed from a 2 mL test at ~100 g/L (in a T-controlled shaker). Samples pulled at time points of 1.5 and 3 h (0.5 mL of the slurry sample) were centrifuged to isolate the solid, and the wet-cake solids were analyzed by XRPD immediately.

XRPD results showed (data not shown) that the conversion process was completed very quickly. At 1.5 h time point, the sample was already converted to anhydrous Form F with high crystallinity.

### 1.3 Characterization of Slurry Conversion Product Post Drying at 40° C.

The wet cake of anhydrous Form F from slurry conversion at 60° C. over 4-day was dried in vacuum oven at 40° C. overnight. The resulting sample was characterized by XRPD, TGA, DSC, PLM and 1H NMR (see FIGS. 5A-5C). XRPD characteristic peaks are summarized in Table 22.

The sample post overnight drying gave the same XRPD pattern as the wet cake, indicating the feasibility of n-heptane removal in vacuum oven at elevated temperature. No residual solvent (either BuOH or n-heptane) was detected by TGA, DSC in the sample post overnight drying at 40° C.

TABLE 22
Observed XRPD peaks for Form F
Pos. [°2 Theta]

7.6
10.8
13.6
14.9
15.4
15.9
17.1
20.5
22.8
22.9

### Example 11

### Preparation and Characterization of Form G

### XRPD

Form G was characterized by XRPD (see FIG. 5A; and Table 23).

TABLE 23
Observed XRPD peaks for Form G
Pos. [°2 Theta]

9.1
12.0
13.5
14.7
16.0
17.2
17.7

What is claimed is:

1. A crystalline form of a Compound 1:

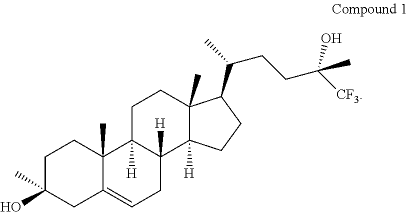

2. The crystalline form of claim 1, wherein the crystalline form of Compound 1 is selected from the group consisting of:
a) crystalline Form A, wherein Form A is characterized by an X-ray powder diffraction (XRPD) pattern comprising peaks at 15.3±0.2, 16.6±0.2, and 17.9±0.2 degrees 2θ;
b) crystalline Form B, wherein Form B is characterized by an XRPD pattern comprising peaks at 14.4±0.2, 16.8±0.2, and 18.5±0.2 degrees 2θ;
c) crystalline Form D, wherein Form D is characterized by an XRPD pattern comprising peaks at 15.2±0.2, 17.4±0.2 and 17.7±0.2 degrees 2θ;
d) crystalline Form E, wherein Form E is characterized by an XRPD pattern comprising peaks at 4.9±0.2, 12.0±0.2, 12.2±0.2, and 17.0±0.2 degrees 2θ;
crystalline Form F, wherein Form F is characterized by an XRPD pattern comprising peaks at 10.8±0.5, 17.1±0.5, 20.5±0.5, and 22.9±0.5 degrees 2θ; and
f) crystalline Form G, wherein Form G is characterized by an XRPD pattern comprising peaks at 9.1±0.5, 14.7±0.5, 16.0±0.5 and 17.2±0.5 degrees 2θ.

3. The crystalline form of claim 1, wherein the crystalline form of the Compound 1 is selected from the group consisting of:
a) crystalline Form A, wherein Form A is characterized by an X-ray powder diffraction (XRPD) pattern comprising peaks at 14.5±0.2, 15.3±0.2, 16.6±0.2, 17.9±0.2, and 20.1±0.2 degrees 2θ;
b) crystalline Form B, wherein Form B is characterized by an XRPD pattern comprising peaks at peaks at 10.9±0.2, 14.4±0.2, 16.8±0.2, 18.5±0.2 and 19.0±0.2 degrees 2θ;
c) crystalline Form D, wherein Form D is characterized by an XRPD pattern comprising peaks at 15.2±0.2, 16.4±0.2, 17.4±0.2, 17.7±0.2, 18.3±0.2, and 18.9±0.2 degrees 2θ;
d) crystalline Form E, wherein Form E is characterized by an XRPD pattern comprising peaks at 4.9±0.2, 12.0±0.2, 12.2±0.2, 17.0±0.2, and 21.4±0.2 degrees 2θ;
crystalline Form F, wherein Form F is characterized by an XRPD pattern comprising peaks at 7.6±0.5, 10.8±0.5, 13.6±0.5, 14.9±0.5, 15.4±0.5, 15.9±0.5, 17.1±0.5, 20.5±0.5, 22.8±0.5, and 22.9±0.5 degrees 2θ; and
f) crystalline Form G, wherein Form G is characterized by an XRPD pattern comprising peaks at 9.1±0.5, 12.0±0.5, 13.5±0.5, 14.7±0.5, 16.0±0.5, 17.2±0.5, and 17.7±0.5 degrees 2θ.

4. The crystalline form of claim 1, wherein the crystalline form of the Compound 1 is selected from the group consisting of:
a) crystalline Form A, wherein Form A is characterized by an X-ray powder diffraction (XRPD) pattern comprising peaks at 11.3±0.2, 14.5±0.2, 15.3±0.2, 16.6±0.2, 17.7±0.2, 17.9±0.2, and 20.1±0.2 degrees 2θ;
b) crystalline Form B, wherein Form B is characterized by an XRPD pattern comprising peaks at 10.9±0.2, 14.4±0.2, 15.4±0.2, 16.8±0.2, 18.5±0.2 and 19.0±0.2 degrees 2θ;
c) crystalline Form D, wherein Form D is characterized by an XRPD pattern comprising peaks at 9.1±0.2, 15.2±0.2, 16.4±0.2, 17.4±0.2, 17.7±0.2, 18.3±0.2, and 18.9±0.2 degrees 2θ;
d) crystalline Form E, wherein Form E is characterized by an XRPD pattern comprising peaks at 4.9±0.2, 8.5±0.2, 12.0±0.2, 12.2±0.2, 14.9±0.2, 15.8±0.2, 17.0±0.2, 18.2±0.2 and 21.4±0.2 degrees 2θ;
e) crystalline Form F, wherein Form F is characterized by an XRPD pattern comprising peaks at 7.6±0.5, 10.8±0.5, 13.6±0.5, 14.9±0.5, 15.4±0.5, 15.9±0.5, 17.1±0.5, 20.5±0.5, 22.8±0.5, and 22.9±0.5 degrees 2θ; and
f) crystalline Form G, wherein Form G is characterized by an XRPD pattern comprising peaks at 9.1±0.5, 12.0±0.5, 13.5±0.5, 14.7±0.5, 16.0±0.5, 17.2±0.5, and 17.7±0.5 degrees 2θ.

5. The crystalline form of any one of claims 1-4, wherein the crystalline form is an anhydrate.

6. The crystalline form of claim 5, wherein the crystalline form is crystalline Form A characterized by an XRPD pattern comprising peaks at 15.3±0.2, 16.6±0.2, and 17.9±0.2 degrees 2θ.

7. The crystalline form of claim 6, wherein the XRPD pattern further comprises at least one additional peak at a position selected from the group consisting of 11.3±0.2, 14.5±0.2, 17.7±0.2, and 20.1±0.2 degrees 2θ.

8. The crystalline form of claim 7, wherein the crystalline form is crystalline Form A characterized by an XRPD pattern comprising peaks at 14.5±0.2, 15.3±0.2, 16.6±0.2, 17.9±0.2, and 20.1±0.2 degrees 2θ.

9. The crystalline form of any one of claims 1-8, wherein the crystalline form is crystalline Form A characterized by an XRPD pattern substantially as depicted in FIG. 1A.

10. The crystalline form of any one of claims 1-9, wherein the crystalline form has a thermo-gravimetric analysis (TGA) thermogram substantially as depicted in FIG. 1B.

11. The crystalline form of any one of claims 1-10, wherein the crystalline form has a differential scanning calorimetry (DSC) thermogram substantially as depicted in FIG. 1B.

12. The crystalline form of claim 11, wherein the DSC thermogram comprises an endothermic peak at about 203° C.

13. The crystalline form of claim 5, wherein the crystalline form is crystalline Form B characterized by an XRPD pattern comprising peaks at 14.4±0.2, 16.8±0.2, and 18.5±0.2 degrees 2θ.

14. The crystalline form of claim 13, wherein the XRPD pattern further comprises at least one additional peak at a position selected from the group consisting of 10.9±0.2, 15.4±0.2, and 19.0±0.2 degrees 2θ.

15. The crystalline form of claim 14, wherein the crystalline form is crystalline Form B characterized by an XRPD pattern comprising peaks at 10.9±0.2, 14.4±0.2, 16.8±0.2, 18.5±0.2 and 19.0±0.2 degrees 2θ.

16. The crystalline form of any one of claim 1-5 or 13-15, wherein the crystalline form is crystalline Form B characterized by an XRPD pattern substantially as depicted in FIG. 2A.

17. The crystalline form of any one of claim 1-5 or 13-16, wherein the crystalline form has a TGA thermogram substantially as depicted in FIG. 2B.

18. The crystalline form of any one of claim 1-5 or 13-17, wherein the crystalline form has a DSC thermogram substantially as depicted in FIG. 2B.

19. The crystalline compound of claim 18, wherein the DSC thermogram comprises an endothermic peak at about 204° C.

20. The crystalline form of any one of claims 1-4, wherein the crystalline form is a solvate.

21. The crystalline form of claim 20, wherein the crystalline form is a hydrate.

22. The crystalline form of claim 21, wherein the crystalline form is a monohydrate.

23. The crystalline form of any one of claim 1-4 or 20-22, wherein the crystalline form is crystalline Form D characterized by an XRPD pattern comprising peaks at 15.2±0.2, 17.4±0.2 and 17.7±0.2 degrees 2θ.

24. The crystalline form of claim 23, wherein the XRPD pattern further comprises at least one additional peak at a position selected from the group consisting of 9.1±0.2, 16.4±0.2, 18.3±0.2, and 18.9±0.2 degrees 2θ.

25. The crystalline form of claim 24, wherein the crystalline form is crystalline Form D characterized by an XRPD pattern with characteristic peaks at 15.2±0.2, 16.4±0.2, 17.4±0.2, 17.7±0.2, 18.3±0.2, and 18.9±0.2 degrees 2θ.

26. The crystalline form of any one of claims 1-4, 20-25, wherein the crystalline form is crystalline Form D characterized by an XRPD pattern substantially as depicted in FIG. 3A.

27. The crystalline form of any one of claims 1-4, 20-26, wherein the crystalline form has a TGA thermogram substantially as depicted in FIG. 3B.

28. The crystalline form of any one of claims 1-4, 20-27, wherein the crystalline form has a differential scanning calorimetry (DSC) thermogram substantially as in FIG. 3B.

29. The crystalline form of claim 28, wherein the DSC thermogram comprises an endothermic peak at about 204° C.

30. The crystalline form of claim 28 or 29, wherein the DSC thermogram comprises an endothermic peak at about 127° C.

31. The crystalline form of claim 20, wherein the crystalline form is an nBuOH solvate.

32. The crystalline form of claim 31, wherein the crystalline form is crystalline Form E characterized by an XRPD pattern comprising peaks at 4.9±0.2, 12.0±0.2, 12.2±0.2, and 17.0±0.2 degrees 2θ.

33. The crystalline form of claim 32, wherein the XRPD pattern further comprises at least one additional peak at a position selected from the group consisting of at 8.5±0.2, 14.9±0.2, 15.8±0.2, 18.2±0.2, and 21.4±0.2 degrees 2θ.

34. The crystalline form of claim 32, wherein the crystalline form is crystalline Form E characterized by having an XRPD pattern with characteristic peaks at 4.9±0.2, 12.0±0.2, 12.2±0.2, 17.0±0.2, and 21.4±0.2 degrees 2θ.

35. The crystalline form of any one of claim 1-4, 20 or 31-34, wherein the crystalline form is crystalline Form E characterized by an XRPD pattern substantially as depicted in FIG. 4A.

36. The crystalline form of any one of claim 1-4, 20 or 31-35, wherein the crystalline form has a TGA thermogram substantially as depicted in FIG. 4B.

37. The crystalline form of any one of claim 1-4, 20, or 31-36, wherein the crystalline form has a differential scanning calorimetry (DSC) thermogram substantially as depicted in FIG. 4B.

38. The crystalline form of claim 37, wherein the thermogram comprises an endothermic peak at about 203° C.

39. The crystalline form of claim 37 or 38, wherein the DSC thermogram comprises an endothermic peak at about 87° C.

40. The crystalline form of claim 5, wherein the crystalline form is crystalline Form F characterized by an XRPD pattern comprising peaks at 10.8±0.5, 17.1±0.5, 20.5±0.5, and 22.9±0.5 degrees 2θ.

41. The crystalline form of claim 40, wherein the XRPD pattern further comprises at least one additional peak at a position selected from the group consisting of 7.6±0.5, 13.6±0.5, 14.9±0.5, 15.4±0.5, 15.9±0.5 and 22.8±0.5 degrees 2θ.

42. The crystalline form of claim 41, wherein the crystalline form is crystalline Form F characterized by an XRPD pattern comprising peaks at 7.6±0.5, 10.8±0.5, 13.6±0.5, 14.9±0.5, 15.4±0.5, 15.9±0.5, 17.1±0.5, 20.5±0.5, 22.8±0.5, and 22.9±0.5 degrees 2θ.

43. The crystalline form of any one of claims 1-4 and 40-42, wherein the crystalline form is crystalline Form F characterized by an XRPD pattern substantially as depicted in FIG. 5A.

44. The crystalline form of claim 5, wherein the crystalline form is crystalline Form G characterized by an XRPD pattern comprising peaks at 9.1±0.5, 14.7±0.5, 16.0±0.5 and 17.2±0.5 degrees 2θ.

45. The crystalline form of claim 44, wherein the XRPD pattern further comprises at least one additional peak at a position selected from the group consisting of 12.0±0.5, 13.5±0.5 and 17.7±0.5 degrees 2θ.

46. The crystalline form of claim 44, wherein the crystalline form is crystalline Form G characterized by an XRPD pattern comprising peaks at 9.1±0.5, 12.0±0.5, 13.5±0.5, 14.7±0.5, 16.0±0.5, 17.2±0.5, and 17.7±0.5 degrees 2θ.

47. The crystalline form of any one of claims 1-4 and 44-46, wherein the crystalline form is crystalline Form G characterized by an XRPD pattern substantially as depicted in FIG. 5A.

48. The crystalline form of claim 1, wherein said crystalline form is selected from the group consisting of:
a) crystalline Form A characterized by an XRPD pattern substantially as depicted in FIG. 1A;
b) crystalline Form B characterized by an XRPD pattern substantially as depicted in FIG. 2A;
c) crystalline Form D characterized by an XRPD pattern substantially as depicted in FIG. 3A;
d) crystalline Form E characterized by an XRPD pattern substantially as depicted in FIG. 4A;
e) crystalline Form F characterized by an XRPD pattern substantially as depicted in FIG. 5A; and
f) crystalline Form G characterized by an XRPD pattern substantially as depicted in FIG. 5A.

49. A solid dosage form comprising the crystalline form of any of the claims 1-48 and at least one pharmaceutically acceptable carrier.

50. A method of treating a CNS-related condition in a subject comprising administering to the subject an effective amount of a crystalline form according to any one of claims 1-48, or solid dosage form according to claim 48.

51. The method according to claim 50, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

52. A method of inducing sedation or anesthesia in a subject, comprising administering to the subject an effective amount of a crystalline form according to any one of claims 1-48, or a solid dosage form according to claim 49.

53. A method of making a crystalline form of Compound 1 comprising the steps of:
a) dissolving Compound 1 in at least one solvent to form a solution;
b) adding an anti-solvent until a precipitate forms; and
c) isolating the precipitate.

54. The method of claim 53, wherein the solvent is selected from the group consisting of THF, 1,4-dioxane and MEK.

55. The method of claim 53 or 54, wherein the anti-solvent is selected from the group consisting of H₂O and ACN.

56. A method of making a crystalline form of Compound 1 comprising the steps of:
a) adding Compound 1 to a solvent to form a mixture;
b) aging the mixture at room temperature to precipitate solids; and
c) isolating the solids.

57. The method of claim 56, wherein the solvent is selected from the group consisting of DME, Acetone, nBuOH, EtOAc, IPAc, BuOAc, diethyl ether, and ACN.

58. A method of making a crystalline form of Compound 1 comprising the steps of:
a) adding Compound 1 to a solvent to form a mixture;
b) aging the mixture at about 40° C. to precipitate solids; and
c) isolating the solids.

59. The method of claim 58, wherein the solvent is selected from the group consisting of IPAc, BuOAc, ACN, and n-heptane.

60. A method of making a crystalline form of Compound 1 comprising the steps of:
a) adding Compound 1 to a volatile solvent to form a mixture;
b) aging the mixture at room temperature to allow the volatile solvent vapor to interact with Compound 1 and to precipitate solids; and
c) isolating the solids.

61. The method of claim 60, wherein the solvent is selected from the group consisting of DME, MEK, diethyl ether and t-BuOH.

62. A method of making a crystalline form of Compound 1 comprising the steps of:
a) adding Compound 1 to solvent to form a solution;
b) adding the solution to an anti-solvent;
c) aging the mixture at room temperature to precipitate solids; and
d) isolating the solids.

63. The method of claim 62, wherein the solvent is BuOH.

64. The method of claim 62 or 63 wherein the anti-solvent is n-Heptane.

65. A method of making a crystalline form of Compound 1 comprising the steps of:
a) adding Compound 1 to solvent to form a mixture;
b) filtering the mixture to provide a filtrate;
c) aging the filtrate at room temperature;
d) allowing solids to precipitate; and
e) isolating the solids.

66. The method of claim 65, wherein the solvent is selected from the group consisting of IPAc, and t-BuOH.

67. A method of making a crystalline form of Compound 1 comprising the steps of:
a) adding Compound 1 to solvent to form a mixture;
b) heating the mixture to about 50° C.;
c) filtering the mixture to provide a filtrate;
d) cooling the filtrate to about −20° C.;
e) allowing solids to precipitate; and
f) isolating the solids.

68. The method of claim 67, wherein the solvent is acetone.

69. A method of making a crystalline form of Compound 1 comprising the steps of:
a) heating Compound 1 to about 205° C.;
b) cooling the heated Compound 1 to about 25° C. to provide solids; and
c) isolating the solids.

70. The method of claim 69, wherein the cooling in step b) is a rate selected from the group consisting of about 2° C./min, about 10° C./min and about 50° C./min.

71. A method of making a crystalline form of Compound 1 comprising the steps of:
a) adding Compound 1 to solvent to form a mixture;
b) aging the mixture at about 90° C. to precipitate solids; and
c) isolating the solids.

72. The method according to claim 71, wherein the solvent is n-heptane.

73. A method of making a crystalline form of Compound 1 comprising the steps of:
a) adding Compound 1 to solvent to form a mixture;
b) grinding the mixture to provide solids; and
c) isolating the solids.

74. The method according to claim 73, wherein the solvent is acetone/MIBK.

75. A method of making a crystalline form of Compound 1 comprising the steps of:
a) adding Form E of Compound 1 to a first solvent to form a mixture;
b) concentrating the mixture by distillation at atmospheric pressure;
c) cooling the mixture to about 80-85° C.;
d) adding a second solvent to the mixture and maintaining the temperature of the mixture at about 80-85° C. for at least 1 hour;
e) concentrating the mixture by distillation at atmospheric pressure;
f) cooling the mixture to about 20° C. to precipitate solids; and
g) isolating the solids.

76. The method according to claim 75, wherein the first solvent is heptane.

77. The method according to claim 75 or 76, wherein the second solvent is n-BuOH.

78. A method of making a crystalline form of Compound 1 comprising the steps of:
a) adding Form E of Compound 1 to a solvent to form a mixture;
b) heating the mixture to about 50° C.;
c) adding antisolvent;
d) adding Form X of Compound 1 seeds and aging the mixture at about 50° C.; e) cooling the mixture to about 20° C. over at least 3 hours and holding the mixture at 20° C. (wet milling optional) to precipitate solids; and
f) isolating the solids.

79. The method according to claim 78, wherein the solvent is acetone.

80. The method according to claim 78 or 79, wherein the anti-solvent is acetone/water.

81. A method of making a crystalline form of Compound 1 comprising the steps of:
a) adding crude Compound 1 to a solvent to form a mixture and heating to about 80° C.;
b) concentrating the mixture via distillation;
c) adding solvent to the mixture;
d) adjusting the temperature of the mixture to about 80° C.;
e) adding Form E of Compound 1 seeds;
f) cooling the mixture to about 15-25° C.;
g) aging the mixture at about 15-25° C. for about 4-18 hours to precipitate solids;
and
h) isolating the solids.

82. The method of claim 81, wherein the solvent is n-BuOH.
